(19) United States
(12) Patent Application Publication
(10) Pub. No.: US 2012/0070338 A1
(43) Pub. Date: Mar. 22, 2012

(54) METHOD AND APPARATUS FOR KILLING MICROBES ON SURFACES WITH AN APPLIED ELECTRIC FIELD

(76) Inventors: Todd R. Schaeffer, St. Michael, MN (US); Thomas R. Denison, Newport Coast, CA (US)

(21) Appl. No.: 13/239,032

(22) Filed: Sep. 21, 2011

Related U.S. Application Data
(60) Provisional application No. 61/384,992, filed on Sep. 21, 2010.

(51) Int. Cl. A61L 2/03

(52) U.S. Cl. 422/22; 422/186.05

(57) ABSTRACT

An apparatus for emitting a controlled electric field upon a microbe-containing surface, and method of use thereof. The apparatus includes a control board and an electric field emitting component. The control board is configured to transmit an electric current to the emitting component, causing an electric field to be emitted therefrom. The electric field is of sufficient strength such that, when the emitting component of the apparatus is positioned proximate the microbe-containing surface, the electric field causes irreversible permeabilization of the cell membrane of microbes on the microbe-containing surface.

### CROSS-REFERENCE TO RELATED APPLICATION

This application claims priority to U.S. Provisional Application No. 61/384,992 filed Sep. 21, 2010, entitled “METHOD AND APPARATUS FOR KILLING MICROBES ON SURFACES WITH AN APPLIED ELECTRIC FIELD”, the entire content of which is hereby incorporated herein by reference in its entirety.

### TECHNICAL FIELD OF THE INVENTION

The present disclosure relates to the destruction of microorganisms on surfaces with the use of an applied electric field. In particular, the present disclosure relates to an apparatus which projects an electric field upon a surface in a manner sufficient to kill microorganisms located thereon, and methods of use thereof.

### BACKGROUND OF THE INVENTION

Experimental research has been conducted on the lethal effect that electrical currents and electrical fields have on microscopic organisms (microbes), including various types of bacteria, mold, viruses and spores. In some of this research, the organisms have been placed between parallel plate electrodes, present in a liquid (for example, in a juice that would normally undergo pasteurization) or if present on a surface, an intermediate substance with electro-conductive properties has been used, for example, water, to transfer or propagate the electric current or electric field to the microbes.

Several mechanisms have been proposed to account for the lethality of electrochemical exposure on microbial cells. These include oxidative stress and cell death due to electrochemically generated oxidants, electrochemical oxidation of vital cellular constituents during exposure to electric current or induced electric fields, and irreversible permeabilization of cell membranes by the applied electric field, also known as irreversible electroporation. These mechanisms are described in greater detail below.

### Chemical Oxidation

Some prior art devices use chemical oxidation to destroy microbes. In these devices, chemical oxidants are generated when electric current is applied to microbes (whether in aqueous suspensions with immersed electrodes or when in direct contact with electrodes). Electrolysis at the electrodes generates a variety of oxidants in the presence of oxygen, including hydrogen peroxide and ozone, as well as free chlorine and chlorine dioxide when chloride ions are present in the solution (for example, if tap or other non-distilled water is used). Such oxidants may also be created within the cells of microbes (in much smaller concentrations) due to the transmittal of electric current throughout region of the microbes being electrically impacted.

This effect has been demonstrated in various experimental forms. FIG. 1 shows a prior art experimental configuration 100 where two electrodes 101, 102 (a cathode and an anode) have been inserted into an aqueous solution 105 containing various strains of microbes. The electrodes are connected to an electric power source 110 such that a current is induced between the electrodes, thus creating an electric charge throughout the solution 105. The electrodes and the electric charge created within solution causes oxidants to form, both intra- and extra-cellularly, leading to eventual cell death.

In another prior art example of a configuration for electro-chemical oxidation of microbes, depicted as FIG. 2, an embodiment of a water spray bottle 200 is shown including a water reservoir 201, an electrolysis cell 210 that includes an ion-exchange membrane suitable for created chemically oxidative water species inside the reservoir 201 configured to induce an electric current in the water and thereby form oxidant chemicals therein, and pump 230 configured to draw water from the reservoir 201 to a spray nozzle 240, and one or more batteries 220 operably connected to both the cell 210 and the pump 230 for providing electric power thereto.

In operation, the spray nozzle 240 of the spray bottle 200 dispenses the electrochemically-activated liquid as a ionized output spray 202. Electrode 245 adjacent nozzle 240 emits an electric field, and the spray apparently provides a path for some of the field to reach the desired surface.

A fuller description of the spray bottle device 200 is given in U.S. patent application Ser. No. 12/639,628 (filed Dec. 16, 2009; published as U.S. 2010/0147700) and Ser. No. 12/639,622 (filed Dec. 16, 2009; published as U.S. 2010/0147701), the contents of which are herein incorporated by reference in their entirety.

### Irreversible Electroporation

The second mechanism of microbial cell death, as mentioned above, is irreversible permeabilization of cell membranes by the applied electric field, also known as irreversible electroporation. In this process, a microbial cell is exposed to an electric field. As a consequence of this exposure, the external portion of the cell membrane gathers charge much like a capacitor, and a trans-membrane potential is induced. A short-lived current across the membrane is established when the membrane is fully charged, demonstrating an induced permeability of the membrane to hydrophilic molecules. In order to deliver an electric field to the microbial cell, an electrode may be placed physically near the cell, or, alternatively, the cell may be in a medium that allows the electric field to be easily carried to it.

Two parameters influence the reversibility of this electropermeabilization: the magnitude of the induced trans-membrane potential, and the duration of the exposure to the external electric field. For microbial cells, trans-membrane potentials above 1 Volt (V) and longer electric pulse times (for example, greater than 0.1 seconds) lead to irreversible permeabilization and cell death. The trans-membrane potential induced by an external electric field depends upon the radius of the cell membrane, with larger cells suffering a greater trans-membrane potential from a given electric field. Cell death occurs due to either the formation of permanent pores and subsequent destabilization of the cell membrane, or loss of important cell components and destruction of chemical gradients via transport through transient pores.

Referring again to the prior art configuration shown in FIG. 1, an electric field 120 has been found to develop about electrodes 101, 102, and propagates through the aqueous medium 105 to come in contact with microbial cells. A trans-membrane potential is induced on the surface of the cells exposed to the field, and cell death follows if the potential and the exposure time to a field formed in this manner are sufficient, as discussed above.

Furthermore, referring again to prior an spray bottle device of FIG. 2, the electrical charge delivered through the liquid 201 dispensed by the spray bottle device 200 is further enhanced by a separate electrode 245 to impart an electrical potential in a liquid output spray and/or stream. The electrode 245, operably connected to the battery 220, is positioned in the liquid path to cause a separate electrical potential as compared to the potential generated by chemical electrolysis cell 201. The electrical potential and associated electric field is thus transmitted via the water spray to the surface 204, where a trans-membrane potential may be induced in microbes located on the surface 204, which, if imparted at a high enough level for a great enough duration of time, will lead to cell death, as discussed above. This device is not capable of delivering an electric field to a surface without the use of a liquid stream and presumably can deliver the field only where the stream forms a continuous path.

### BRIEF SUMMARY OF THE INVENTION

Disclosed herein, in one embodiment, is an apparatus for emitting a controlled electric field for selective killing of microbes, which may include a control circuit, connectable to a power source, and comprising a current waveform generating component, wherein the control circuit receives an input electric current from the power source, and wherein the current waveform generating component transforms the input electric current into an output electric current with a predetermined waveform; and an electric field emitting component, for receiving output electric current from the control circuit, comprising at least one emitter for emitting an electric field, wherein the pulse interval generating component transmits the output electric current from the control circuit to the emitter, thereby causing a controlled electric field to be emitted from the emitter with a predetermined waveform, sufficient to cause irreversible permeabilization of a cell membrane of microbes on the electric field emitting component or on a microbe-containing surface proximate to the electric field emitting component.

Disclosed herein, in a further embodiment, is a hand-held apparatus for killing microorganisms on a microbe-containing surface, which may include a body portion; a user control component positioned on an exterior surface of the apparatus a control circuit, connected to the user control component; and a head portion, extending from the body portion, connected to the control circuit, and comprising an emitter on an electric field-emitting surface thereof, wherein actuation of the user control component causes the control circuit to transmit an electric current to the emitter, thereby causing the emitter to emit an electric field from the electric field-emitting surface, sufficient to cause irreversible permeabilization of a cell membrane of microbes on a microbe-containing surface proximate to the head component.

Disclosed herein, in a further embodiment, is a method for killing microorganisms on a microbe-containing surface using a controlled electric field, which may include providing a head component comprising an array of emitters on an electric field-emitting surface thereof; providing a control circuit comprising an actuator, electrically connected to the head component, and configured such that when the actuator is actuated, the control circuit transmits an electric current having a voltage waveform to the emitters at a pulse interval; positioning the head component such that the electric field-emitting surface is facing toward and positioned proximate to a microbe-containing surface; and actuating the actuator, thereby causing the controlled electric field to be emitted from the electric field-emitting surface and toward the microbe-containing surface, and wherein the electric field causes irreversible permeabilization of a cell membrane of microbes on the microbe-containing surface.

Disclosed herein, in a further embodiment, is an apparatus for emitting a controlled electric field onto a microbe-containing surface, which may include a control circuit, connectable to a power source, and an AC power generating component, wherein the control circuit receives an input electric current from the power source transforms the input electric current into an output electric current having a fundamental frequency; and an emitter connector component, for receiving current from the control circuit, and delivering it to at least one emitter for emitting an electric field, wherein the control circuit transmits the output electric current from the emitter connector to the emitters at a fundamental frequency in the range from 10 KHz to 200 KHz and subject to over-current control, thereby causing a controlled electric field to be emitted from the emitters with a defined waveform, sufficient to cause irreversible permeabilization of a cell membrane of microbes on a microbe-containing surface proximate to the head component.

In variations of this embodiment, the emitter connector may connect to an array of emitters mounted on a flexible substrate. The flexible substrate may be a surface on a glove. The emitter connector may connect to a head component comprising a field transport layer that facilitates delivery of the electric field to the microbe-containing surface.

In further variations, the field transport lay may include a variety of materials. In particular, the field transport layer may include a material resilient and deformable to follow contours of the microbe-containing surface. The field transport layer may include a wiping cloth removably attached to the head component. The field transport layer may include a material porous and capable of holding a cleaning solution. The field transport layer may include a colloid with a permittivity of 30 or greater. The field transport layer may include a hydrocolloid with a permittivity of 30 or greater. The field transport layer may include a material resilient and deformable to follow contours of the microbe-containing surface with a friction-reducing outer layer. The field transport layer may include a material resilient and deformable to follow contours of the microbe-containing surface with a friction-reducing outer layer that may include a wiping cloth. Further, the field transport layer may include a material resilient and deformable to follow the contours of the microbe-containing surface.

In further variations of the embodiment, the head component may include stand-off projections to separate the array of emitters from direct contact with the microbe-containing surface, said projections being made of a low friction material. The stand-off projections may be positioned at the periphery of the head component and the low friction material is a hard, low friction resin. The hard, low friction resin may be selected from the following group: a nylon, resin, and acetal. The apparatus of claim 1, wherein the emitter connector detachably connects to a component to be treated for microbes, said component being capable of functioning as an emitter so as to deliver the controlled electric field essentially simultaneously to all points on the component.

In further variations, the component to be treated for microbes is a working surface. The component to be treated for microbes may be a cover layer for a working surface. The component to be treated for microbes may be a curtain.

Disclosed herein, in a further embodiment, is a method for killing microbes, which may include providing an electrically conductive emitter for emitting an electric field for killing microbes in contact with or in close proximity to the emitter; and providing a control circuit for electrical connection to the emitter to deliver a current with an AC pulse waveform having a fundamental frequency in the range of 10 KHz to 200 Hz; said control circuit being activated to deliver the current for a defined interval, causing the emitter to emit an electric field sufficient to cause electroporation of microbes in contact with or in close proximity to the emitter, said current being controlled to a level that limits arcing from the emitter to adjacent objects.

In variations of this embodiment, the step of providing the emitter may include providing an emitter selected to conform to a surface to be treated. The step of providing the emitter may include providing an emitter that is conformable into intimate contact with a portion of a surface to be treated. The step of providing the emitter may include providing an emitter consisting of an array of separate emitters on a substrate conformable into intimate contact with a portion of a surface to be treated. The step of providing the emitter may include providing an emitter including a conductive portion that is deformable. Further, the step of providing the emitter may include providing an emitter including a conductive portion and a deformable field transport layer with a relatively high permittivity.

The various embodiments have in common the ability to deliver to a variety of target surfaces (flat, curved or irregular, smooth or rough, hard or soft, and of a variety of materials) an electric field sufficient to destroy microbes located on such surface, without requiring a flow of or flooding with water or other liquid or fluid (including air). In some embodiments, the field is applied with no liquid or other substance introduced to the target surface. In practice, water may be applied to a surface to help lift dirt from a surface or otherwise facilitate the cleaning and removing of dirt, but that water is not needed as a conductive path and not relied on to kill microbes.

While multiple embodiments are disclosed, still other embodiments of the present disclosure will become apparent to those skilled in the art from the following detailed description, which shows and describes illustrative embodiments. As will be realized, the invention is capable of modification in various aspects, all without departing from the spirit and scope of the present disclosure. Accordingly, the drawings and detailed descriptions are to be regarded as illustrative in nature, and not restrictive.

### BRIEF DESCRIPTION OF THE FIGURES

While the specification concludes with claims particularly pointing out and distinctly claiming the subject matter that is regarded as forming the various embodiments of the present disclosure, it is believed that the embodiments will be better understood from the following description taken in conjunction with the accompanying Figures, in which:

FIG. 1 is a prior art experimental configuration for delivering electric current to an aqueous solution containing microbes using a pair of electrodes.

FIG. 2 is a prior art water spray bottle device configured with an electric cell to deliver an electric charge to a surface from the bottle using the water spray as a transient electrically conductive medium.

FIGS. 3A-B depict an emitter in the form of a wire, the latter figure having an insulation material surrounding a portion thereof.

FIGS. 4A-B depict the electric field which is formed about an un-insulated portion of wire when current is applied thereto.

FIGS. 4C-D depict the electric field which is formed about an insulated portion of wire when current is applied thereto, the former being insulated only on one side of the wire along the entire length of the wire, while the latter being insulated fully but only about a portion of the wire length.

FIG. 4E depicts the electric field which is formed about a fully insulated portion of wire when current is applied thereto, the insulating material being sufficiently permittive to allow an electric field to be propagated therethrough.

FIGS. 5A-B depict an emitter in the form of a microstrip electrode, the former being a plan view of the field emitting surface, the latter being a side elevation view thereof.

FIGS. 6A-B depict the electric field which is formed about the microstrip electrode of FIGS. 5A-B.

FIG. 7 depicts a side view of an electric field-emitting head component in accordance with the present disclosure.

FIGS. 8A-B depict an example circular emitter head component having a plurality of wire emitters in a frontal view of the electric field-emitting surface, and a pictorial view, respectively.

FIG. 9 depicts an example emitter head component in a brush configuration having a plurality of partially-insulated wire emitters.

FIGS. 10A-B depict an example circular emitter head component having a plurality of micro-strip electrodes in a frontal view of the electric field-emitting surface, and a pictorial view, respectively.

FIGS. 11A-B depict an example emitter head component having a plurality of square micro-strip electrode emitters in a frontal view of the electric field-emitting surface, and a pictorial view, respectively.

FIG. 12A depicts an example emitter head component in a cloth or flexible sheet configuration having a plurality of micro-strip electrode emitters.

FIG. 12B depicts an example head component configured to have an emitting fabric or flexible sheet affixed thereto.

FIG. 13 depicts an example emitter head component in a glove configuration having a plurality of micro-strip electrode emitters.

FIGS. 14A-B depict an example circular emitter head component having a plurality of long-strip emitters in a frontal view of the electric field-emitting surface, and a pictorial view, respectively.

FIGS. 15A-B depict an example square emitter head component having a plurality of long-strip emitters in a frontal view of the electric field-emitting surface, and a pictorial view, respectively.

FIGS. 16A-B depict an example emitter circular head component having a single long-strip emitter in a frontal view of the electric field-emitting surface, and a pictorial view, respectively.

FIG. 17A-D depict example emitter head components with electric field propagation-enhancement/damage prevention components in the form of protective spacers, resilient contact layer, disposable contact layer, and a resilient contact layer/low friction contact layer composite, respectively.

FIGS. 18A-B depict example pulse waveforms generated by a control board in accordance with the present disclosure.

FIG. 18C is a block diagram of a control and driver board in accordance with the present disclosure.

FIG. 19 depicts an example of a surface to be treated that is irregular and has crevices, illustrating how one embodiment in accordance with the present disclosure permits an electric field to be delivered with microbe-killing effect.

FIG. 20 shows in schematic form an experimental configuration of an apparatus in accordance with the present disclosure.

FIGS. 21A-D depict example microbe-containing surfaces used in an experiment with the configuration of FIGS. 20A-D, wherein FIGS. 21A and 21C show the surfaces before the use of the apparatus, and FIGS. 21B and 21D show the surfaces after the use of the apparatus.

FIGS. 22A-B show alternative configurations of an example hand-held electric field-emitting apparatus in accordance with the present disclosure, FIG. 22B including a reservoir.

FIG. 23 shows a wand-shaped, “duster” embodiment of a hand-held electric field-emitting apparatus in accordance with the present disclosure.

FIGS. 24A-24C schematically depict examples of apparatus with a separable emitter component connected therewith in accordance with the present disclosure.

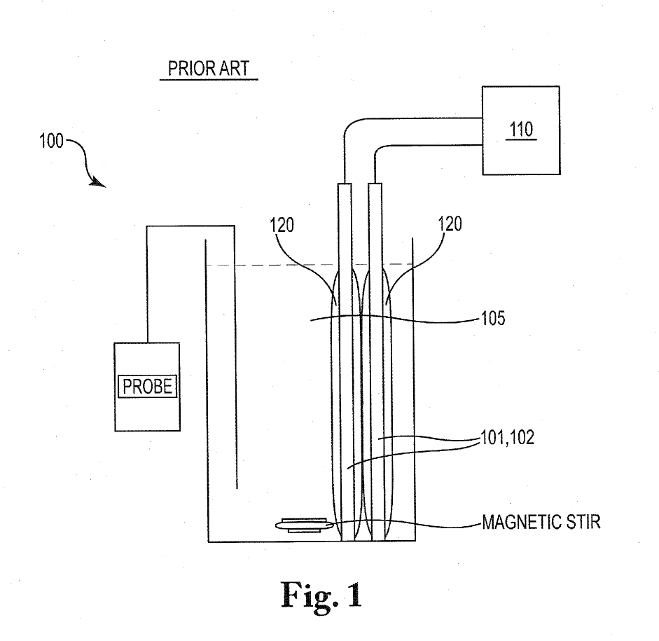

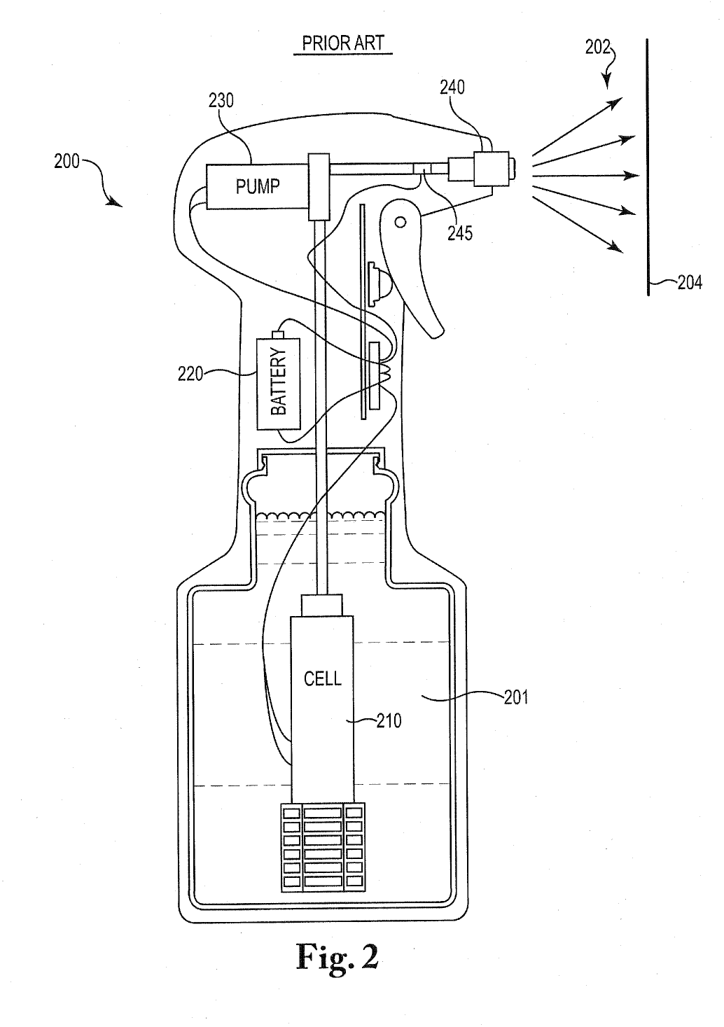

### DETAILED DESCRIPTION OF THE INVENTION

As used herein, the term “microbe” means microorganisms selected from the group consisting of bacteria, viruses, yeasts, fungus, spores, and combinations of any of the foregoing In some embodiments, microbes are bacteria or viruses or combinations of bacteria and viruses. In some embodiments, microbes are bacteria.

### Overview of Electric Field Theory

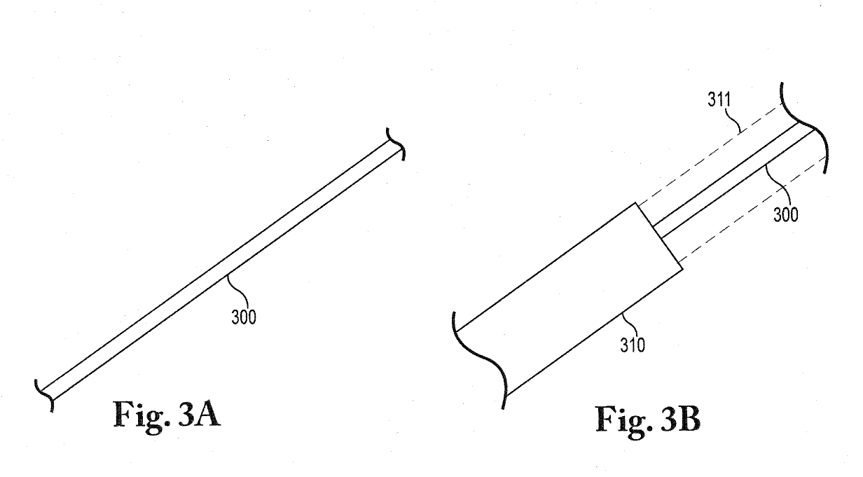

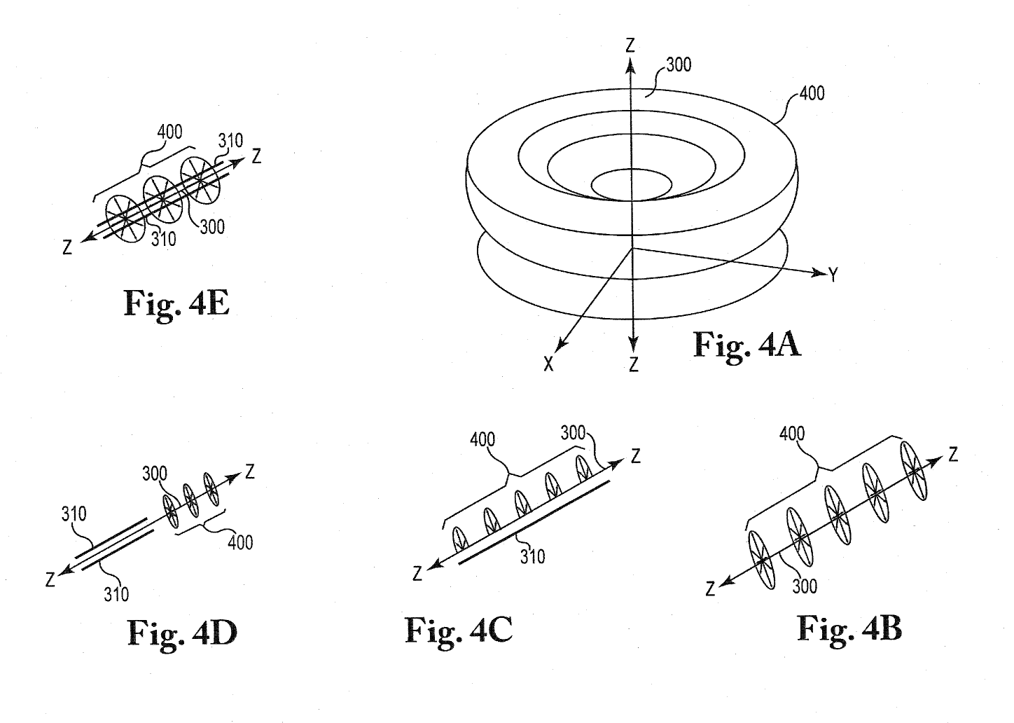

Electrically conductive plates, leads, strips, wires, conductive fabric or electrodes, hereinafter referred to generally as “electric field emitters”, or more simply “emitters”, generate an electric field when charged with electric current. The shape of the electric field depends on the shape of the charged emitter. The electric field can be transmitted through many materials. For example, FIG. 3A depicts an emitter in the form of a wire 300, which may be a copper wire, or a wire made of any other electrically conductive metal such as silver, tin, aluminum, etc., or any other electrically conductive material, such as a charged polymer matrix. When electricity is conducted through the wire in the form of electric current, an electric field is generated about the wire in a generally toroidal shape. FIG. 4A depicts the general toroidal shape of an electric field 400 about a small portion of charged wire 300 extending along the Z-axis (aligned with the length of the wire), while FIG. 4B represents the electric field as it would appear about a longer piece of wire 300, also along the Z-axis.

The distance that an electric field extends from an emitter depends on the electrical permittivity and electric field strength (based on current and voltage), designated in the art by the symbol ε, of the environment surrounding the emitter. Environments with high permittivity, such as aqueous solutions with a high ion concentration, have a relatively high permittivity, while insulating materials, such as various plastics, rubbers, and other long-chain organic compounds have a relatively low permittivity. Thus, the strength of the electric field about an emitter can be influenced by its surrounding environment. Additionally, the proximity of an emitter to the target surface affects the electric field (field strength generally decreases with distance from the point of radiation), even in the absence of a high permittivity medium. For example, an emitter placed very near a surface, with no intervening material other than air, may deliver a strong electric field to such surface.

Because field strength increases the ability to kill microbes or decreases the exposure time required to kill, it is desirable for an emitter to achieve contact or the greatest proximity possible to the target microbes, consistent with possible undesired arcing to a surface that could be damaged by arcing. Accordingly, the emitters described herein are designed to contact or achieve close proximity with the surface where microbes to be killed may be located. However, surfaces on which microbe killing is desired are not always flat and totally smooth, i.e., almost always have microbe-accommodating crevices, making the contact or close proximity difficult to achieve. One problem addressed by the various embodiments described herein is how to bring emitters or portions thereof into contact or close proximity with target surfaces while controlling undesired arcing to a target surface or shorting between emitter elements, both of which may adversely affect projection of the electrical field over the desired area, and may cause damage.

Applying an insulating material about a portion of an emitter to help control the direction and/or strength of the generated electric field to suit the needs of the particular application is one control approach. The present invention contemplates use of a variety of field emitters and use of a variety of environments, with the goal of effectively delivering to flat, curved, irregular, smooth or rough surfaces in close proximity to the emitter a field of sufficient strength to reduce or essentially kill microbes present on such surfaces. Accordingly, the emitters may be larger and generally planar where a surface to be treated is large and planar, or, to permit irregular surfaces to be treated, the emitter may include an array of smaller emitters, deployed on a substrate that may be flexed or deformed to allow at least a portion of an array of emitters to substantially conform to an irregular surface. Further, a flexible substrate may be a continuous conductive material, such as a conductive fabric, whereby a number of smaller emitters are merged into a continuous or nearly continuous emitting surface or thin layer. For example, as shown in FIG. 3B, the portion of electrically conductive wire 300 of FIG. 3A has been surrounded on a portion thereof with an insulating material 310 having a relatively low electrical permittivity or a relatively high electrical permittivity. Alternatively, the entire wire 300 is surrounded by an insulating material (indicated by dashed lines 311). In this alternative, the insulating material may have a selected, relatively high electrical permittivity, thereby allowing an electric field to be propagated from the wire 300, while preventing current to pass therethrough to prevent electric shorting or arcing. FIGS. 4C and 4D show the resulting electric fields 400 about wires having alternative configurations of insulation. FIG. 4C shows the resulting electric field 400 about a wire 300 having an insulating, low permittivity material 310 disposed along the entire length of the wire, but only on one side of the wire. As shown, the electric field 400 propagates in a generally half-cylinder formation about the exposed (non-insulated) side of the wire 300. In contrast, as shown in FIG. 4D (which shows the resulting field from the wire/insulation configuration of FIG. 3B), the electric field 400 is emitted in the generally cylindrical shape about the un-insulated portion of the wire 300, while the insulated portion emits an electric field of lesser magnitude (negligible, if the insulation is highly effective or even acts as shielding).

FIG. 4E illustrates the fully insulated configuration of FIG. 3B. A wire emitter 300 is fully insulated (or “shielded”) with insulating material 310 to substantially prevent conduction of electric current. However, the insulating material in this example is electric field permittive—that is, the electrical permittivity is still sufficiently high that an electric field 400 propagates through the insulating material 310. Such materials are known in the art, the selection of which depend the insulative/permittive qualities desired in the particular embodiment employed.

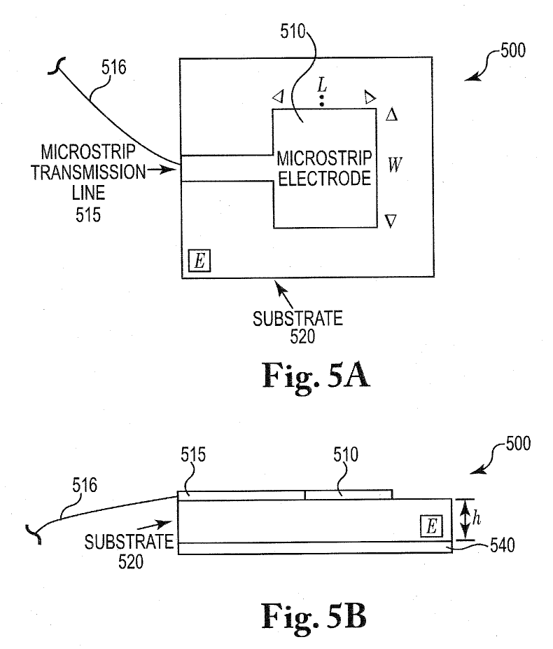

An alternative configuration for an emitter is shown in FIGS. 5A-B. A micro-strip electrode emitter 500 is depicted with the electrode portion 510 being positioned atop a substrate 520 having a low electrical permittivity (e), thereby insulating the undersurface of the electrode 510. The electrode portion 510 has a defined length (L), width (W), which may each range in size from about 1 micrometer to 5 centimeters, or preferably from about 1 millimeter to 1 centimeter. Of course, in theory, various larger or smaller sizes of such electrode would be possible. The electrode portion 510 is supplied with electric current via transmission line 515. The transmission line 515, in turn, may be charged with electric current from a feed line 516, which may be connected to an electric power source. A ground plate 540, as shown in FIG. 5B, may be supplied on the surface of the substrate 520 opposite the electrode portion 510 to prevent any residual electric current not inhibited by the low permittivity substrate material from transmitting beyond said opposite surface of the micro-strip electrode emitter 500. The substrate may have a height (h), which may generally range from about 1 micrometer to 1 centimeter, or preferably from about 0.5 millimeter to 5 millimeters. Of course, as with the dimensions of the electrode portion, a variety of heights (h) of the substrate 520 are possible.

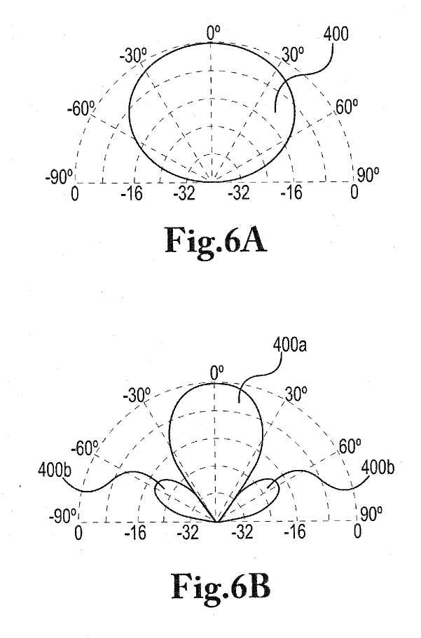

The electric field generated by applying current to the micro-strip electrode emitter of FIGS. 5A-B is depicted in FIGS. 6A-B. In FIG. 6A, showing a view from the width (W) side of the electrode portion 510, the electric field 400 generally appears as an oblong, oval, or “balloon” shape. In a view from the opposite side from the (W), FIG. 6B, the effect of the field generated by the transmission line 515 is apparent. The main field generated by the electrode portion 510 is the larger “balloon” shaped field 400a, while the relatively smaller (and more oblong shaped) fields 400b are generated from either side of the transmission line 515.

In addition to a projected electric field, emitters as described herein may provide an electric current, i.e., a small transfer of charge directly to the surface in question or microbes on it. For example, bringing the emitter in close proximity with an irregular surface may cause an electric field to be emitted generally in the area of proximity to the emitter, while certain points of direct contact with the emitter may be exposed to a electric charge flowing from the emitter. Thus, while the description of the emitters herein speaks mainly in terms of delivery an electric field, it will be understood that some microbe killing may occur as a result of charge transfer to the microbe, not just by reason of field effects. This small amount of charge delivery is acceptable in some environments where it does not cause a fire hazard or damage a surface.

As will be appreciated, the above example emitters, and the resultant electric fields generated when current is applied therethrough, are merely examples, and are not to be interpreted as limiting. Other emitter configurations are possible, which generate electric fields of sufficient strength to kill microbes when the emitter is brought into contact or close proximity with a target surface. All such emitters should be considered within the scope of this disclosure.

### Electric Field-Emitting Head Component

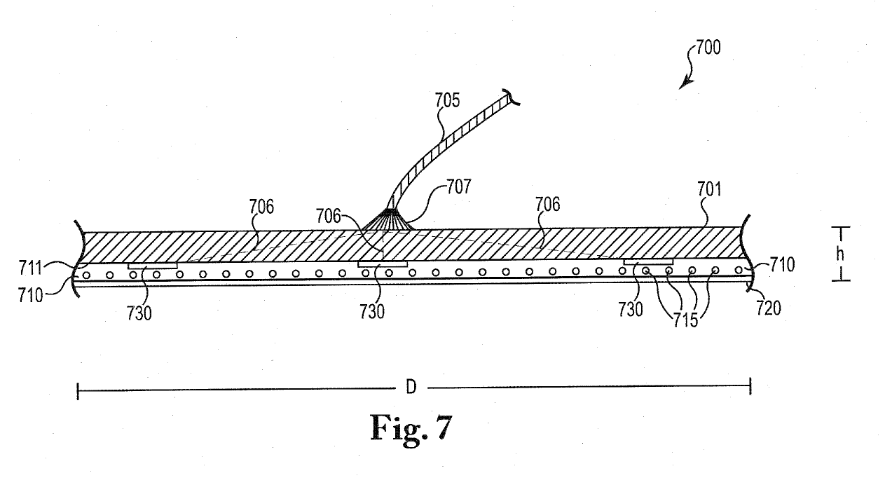

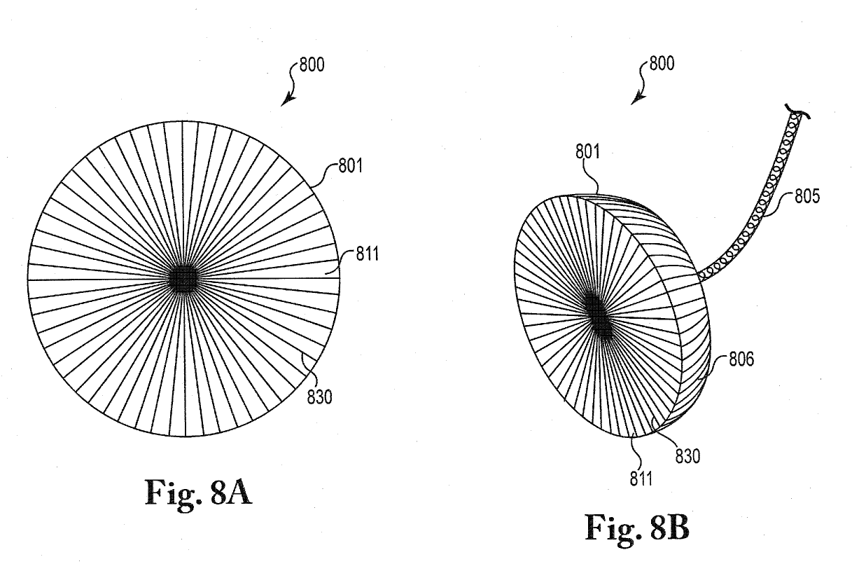

Electric field-emitting head components of the present disclosure are generally designed so as to allow the projection of an electric field of microbe-killing capacity to a microbe containing surface without the aid of liquids or sprays. In one embodiment of the present disclosure, as shown in FIG. 7A, an electric field-emitting head component 700 includes a generally planar head portion 701. The head base-layer 701 may generally be made of a material having low electrical permittivity, as discussed above. Such materials include plastics, rubbers, and other long-chain organic compounds. Alternatively, the head base layer 701 may be made of any material, and covered with, or shielded with, a material having low electrical permittivity, such as electrical tape. In a particular embodiment, the head portion is a plastic disc covered with electrical tape. The head portion 701 may generally be of any shape, such as circular, square, rectangular, oval, etc. If circular, it may generally have a diameter D between about 0.5 cm-1 m, 1 cm-50 cm, or preferably between about 5 cm-25 cm. If rectangular, it may generally have dimensions of about 1 cm-3 cm×1 cm-3 cm, 3 cm-10 cm×3 cm-10 cm, 10 cm-20 cm×10 cm-20 cm, 20 cm-1 m×20 cm-1 m, or 8 in-14 in×2.5 ft-3.5 ft. Other shapes and dimensions are considered to be within the scope of the disclosure. The head portion 701 may generally have a height h between about 0.2 cm-10 cm, or preferably between about 0.5 cm and 5 cm.

On an electric field-emitting surface 711 of the head portion 701, one or more emitters 730 may be present. These emitters, as discussed above, may emit an electric field when supplied with electric current. They may also provide an electric current (a small transfer of charge) directly to microbes on the surface with which portions of the emitter arc brought into direct contact such that the organism's conductivity causes some charge transfer. In some embodiments, electric current may be supplied from a power source (not shown) to the emitter by means of a supply wire 705. The wire 705 may generally extend from the power source (or other component itself connected to a power source) to a connection point 707 on the surface of the head portion opposite the electric field-emitting surface 711. At this connection point 707, the wire may enter the head portion 701 and split off within the head portion 701, such that a wire lead (shown as dotted lines 706) extends through the interior of the head portion 701 to each emitter 730, thereby supplying each emitter with the appropriate electrical field.

In an alternative embodiment, rather than employing split-off wire leads 706 within the head portion 701, the split-off wire leads 706 may extend from the connection point 707 around the exterior of the head portion 701 to the emitters 730 on the electric field-emitting surface 711.

As shown in FIG. 7, the head component 700 may be positioned proximate a surface 720 having a plurality of microbes 715 thereon, with the electric field-emitting surface 711 thereof facing the microbe-containing surface 720. The head component 700 is preferably positioned such that there is essentially direct contact between the electric field-emitting surface 711 (specifically the emitters 730 located thereon) and the microbe-containing surface 720. Depending on the smoothness/roughness of the surface to be treated, the head component 700 may alternatively be brought into direct contact with the surface 720 (or portions thereof) and the microbes on or near the outermost portions of the surface 720.

In one mode of operation, electric current is supplied to the emitters 730 by means of the supply wire 705 and the split-off wire leads 706, with the head portion 701 positioned proximate to the microbe-containing surface 720, with the electric field-emitting surface 711 facing the microbe-containing surface 720. An electric field is generated from the emitters 730, as discussed above, and the emitter is placed directly on the wood, concrete, plastic, ceramic, paper or other surface containing the microbes 715, where the electric field causes irreversible permeabilization (electroporation) of the cell membrane of the microbes 715, killing them (or a high percentage thereof), and thus reducing or destroying the microbial burden on the surface 720. Alternatively, for some surface materials that are more conductive (e.g., a conductive cloth or a paper or cloth coated with a conductive layer), an electric current may be supplied directly to the surface 720 (or portions thereof) so that it or portions of it become an extension of the emitters 730, resulting in microbe death in one of the manners discussed above of microbes in intimate contact with the surface.

Other configurations of the electric field-emitting head component 700 will now be disclosed. The embodiment of FIGS. 8A-B is an electric field-emitting head component 800, of a generally circular shape, and having a plurality of wire emitters (in the manner of FIGS. 3A, 4C) which extend across the electric field-emitting surface 811 and cross at a central point thereof. The supply wire 805 splits-off to a plurality of wire leads extending about the exterior surface of the head portion 801, which connect with and supply electric current to the plurality of wire emitters 830 on the electric field-emitting surface 811. This configuration delivers an electric field or electric charge from each of the plurality of wire emitters 830.

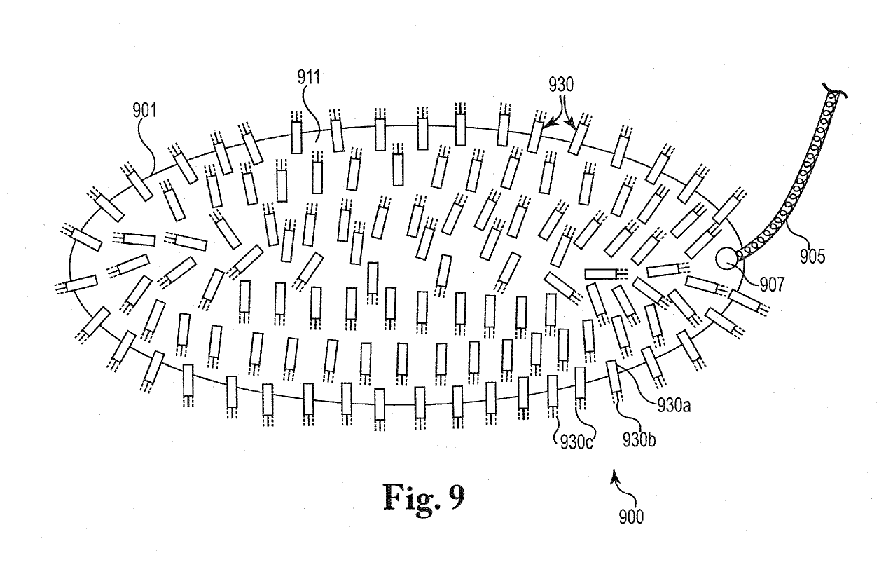

The embodiment of FIG. 9 is an electric field-emitting head component 900, of a “brush” configuration, having a plurality of electric wire emitters 930 which extend from the electric field-emitting surface 911 (which in this embodiment comprises substantially the entire exterior surface of the head portion 901). In this embodiment, the head portion 901 is designed to be “brushed” across a microbe-containing surface. So as to prevent the wire emitters 930 from touching each other during the brushing motion (and thereby potentially causing a short), the emitters are short and lower portions of each emitter 930 have been insulated in the manner of FIG. 3B and FIG. 4D, discussed above. As shown, insulation 930a covers the wire portion of the emitter 930 from where the wire emitter 930 abuts the surface 911 to approximately half way along the extended wire. The upper portion of the emitter 930b, in one alternative embodiment, is un-insulated, and is therefore able to project an electric field. Alternatively, each emitter is un-insulated and extends a short distance above the surface 911. In a further alternative, each emitter is insulated along its full length and optionally at its distal end (indicated as dashed lines 930c), wherein the insulating material is sufficiently insulative to prevent electrical current from conducting therethrough, yet has a high enough electrical permittivity to allow a sufficient electric field to be emitted. Such electrically insulative yet permittive materials are known in the art, the selection of which being dependent upon the particular target surfaces and surface configurations, the goal being to avoid significant arcing to a surface while still delivering a field or limited charge of sufficient strength to destroy microbes. Each emitter is supplied with electric current (and a corresponding field) by means of supply wire 905, which splits-off at connection point 907 into a plurality of split-off wire leads (not shown) within the interior of the head portion 901, to connect with each emitter 930. This configuration delivers an electric field or charge from each of the plurality of emitters 930.

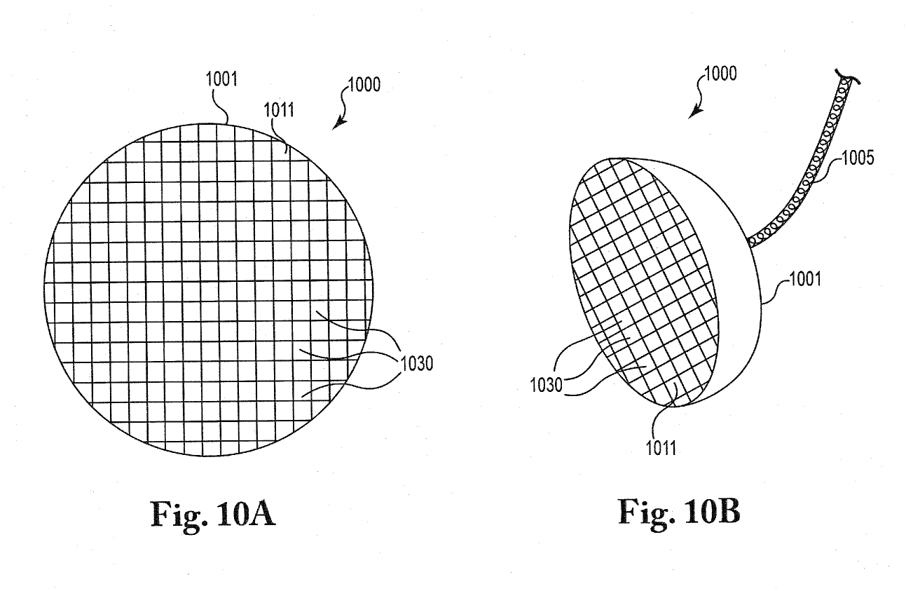

The embodiment of FIGS. 10A-B is an electric field-emitting head component 1000, of a generally circular shape, and having a plurality of micro-strip electrode emitters (in the manner of FIGS. 5A-B, 6A-B) which are positioned across the electric field-emitting surface 1011 in a grid-like pattern. The supply wire 1005 splits-off to a plurality of wire leads extending through the interior of the head portion 1001 (not shown), which connect with and supply electric current to the plurality of micro-strip electrode emitters 1030 on the electric field-emitting surface 1011. This configuration delivers an electric field or charge from each of the plurality of micro-strip electrode emitters 1030.

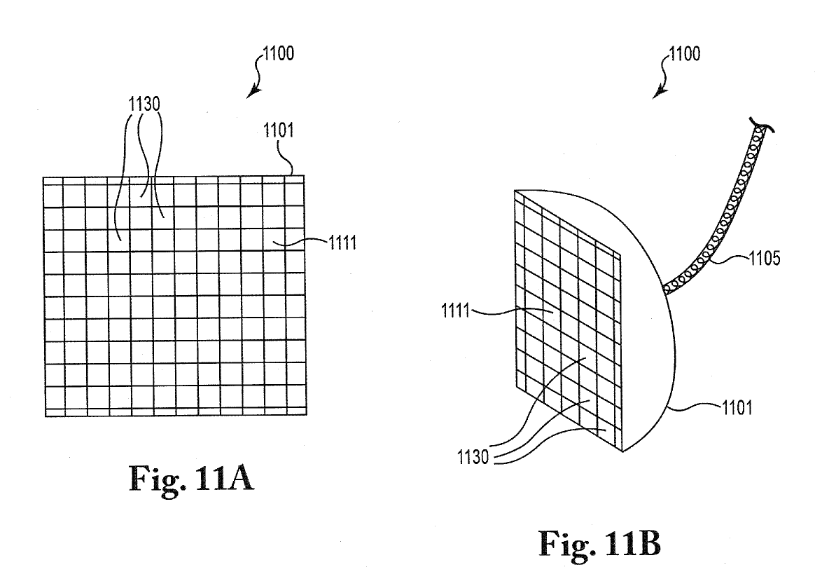

The embodiment of FIGS. 11A-B is an electric field-emitting head component 1100, of a generally square or rectangular shape, and having a plurality of micro-strip electrode emitters (in the manner of FIGS. 5A-B, 6A-B) which are positioned across the electric field-emitting surface 1111 in a grid-like pattern. The supply wire 1105 splits-off to a plurality of wire leads extending through the interior of the head portion 1101 (not shown), which connect with and supply electric current to the plurality of micro-strip electrode emitters 1130 on the electric field-emitting surface 1111. This configuration delivers an electric field or charge from each of the plurality of micro-strip electrode emitters 1130.

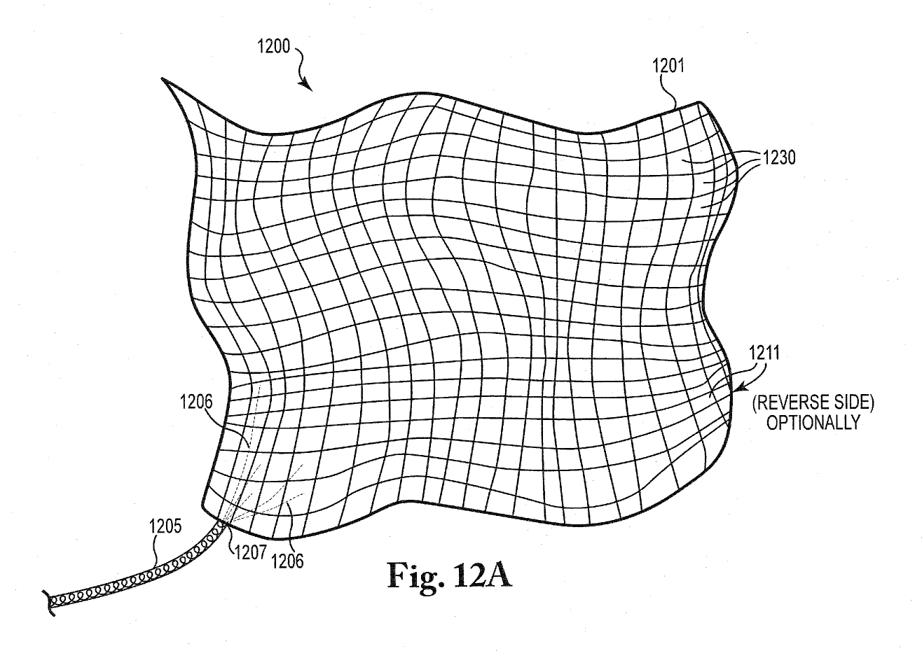

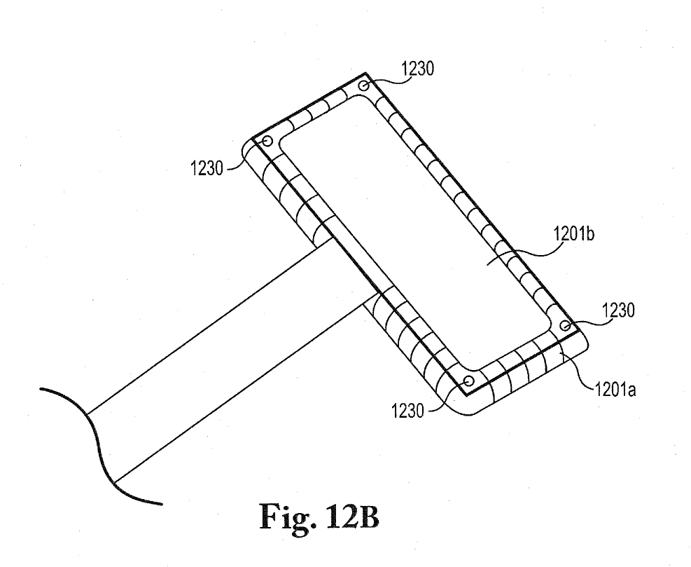

The embodiment of FIG. 12A is an electric field-emitting head component 1200, in the configuration of a cloth or a flexible substrate, having a plurality of micro-strip electrode emitters (in the manner of FIGS. 5A-B, 6A-B), which are positioned across the electric field-emitting surface 1211 (which in this embodiment may be two surfaces—both sides of the cloth) in a grid-like pattern. Alternatively, thin wire emitters may be woven directly into, or otherwise embedded in spaced relation to each other in, the cloth or other flexible substrate material. The head portion 1201 may be made of a low-electric permittivity and non-conductive cloth-like material, such as a synthetic fiber or a synthetic polymer, among others. The emitters 1230 may be secured to the surface 1211 of the cloth head portion 1201 by stitching, gluing, or any other form of secure affixing. The emitters 1230 and associated wiring also may be placed by printing processes on a layer of flexible material. The emitters 1230 may be covered by an insulating layer, so that accidental contact between individual emitters and resultant undesired shorting may be prevented or reduced. Supply wire 1205 extends to the cloth “emitter head” portion 1201, connecting thereto at connection point 1207, where it splits-off into lead wires 1206 (shown as dotted lines), which connect with and bring electric current to each micro-strip electrode emitter 1230. Split-off lead wires 1206 may be woven within the cloth, placed in between two layers of cloth-like material or otherwise extend within the interior of the cloth material, to reach each emitter. This configuration delivers an electric field from each of the plurality of micro-strip electrode emitters 1230. In typical operation, the head component 1200 of FIG. 12 may be gripped by a bare-handed user on the non-electric field emitting surface (the surface with no emitters) and wiped across a microbe-containing surface in the manner of using a wiping-cloth so as to project an electric field onto said surface to kill the microbes located thereon.

An alternative embodiment is depicted in FIG. 12B. In this embodiment, the cloth portion 1201a is itself an electrically conductive material, and as such is capable of delivering an electric field to whatever surface it may be applied. An example fabric suitable for use with the present disclosure is the MedTex130™ Conductive Fabric supplied by SparkFun Electronics of Boulder, Colo. The cloth is silver-plated nylon that is stretchy in both directions. It is conductive with a surface resistivity of <1 ohm/sq. This example fabric has a thickness of 0.45 mm, and a weight of 140 g/m². An alternative cloth is the MedTex180™, which is slightly thicker and heavier, at 0.55 mm and 224 g/m².

The example of FIG. 12B shows the conductive cloth 1201a attached to a head 1201b that includes one, two, three, or more emitter components 1230a. These emitters electrically contact the cloth 1201a, which in turn projects an electric field and at some points of contact transfers charge to the surface to which it is applied. Attachment of the cloth may be made by any suitable means, including adhesives, clips, straps, and the like. The cloth may be disposable or washable, wherein the user replaces or washes the cloth 1201 after a period of use.

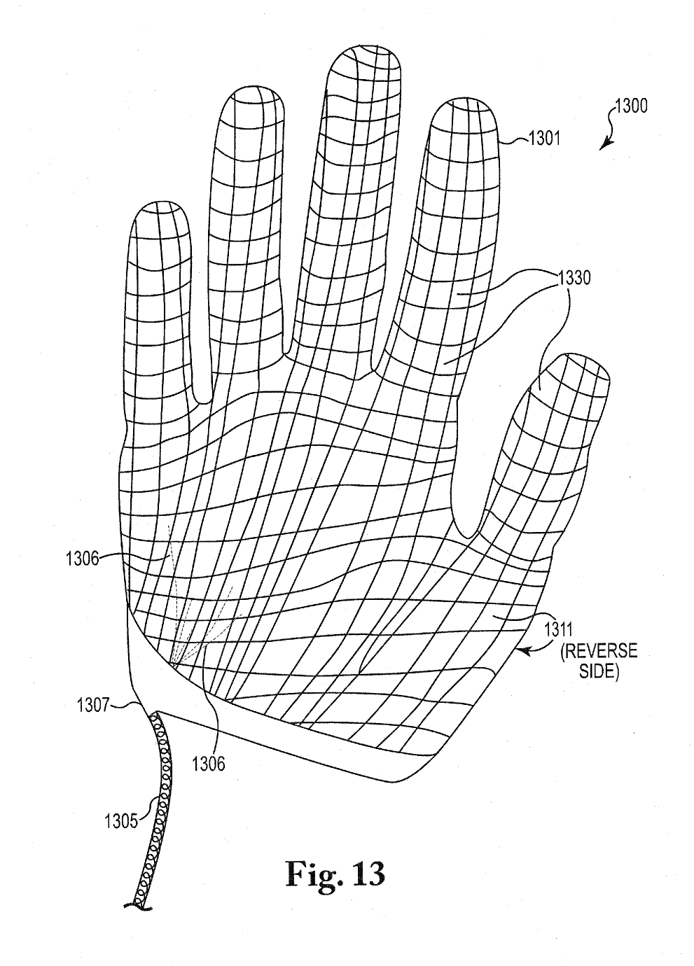

The embodiment of FIG. 13 is an electric field-emitting head component 1300, in the configuration of a glove, having a plurality of micro-strip electrode emitters (in the manner of FIGS. 5A-B, 6A-B) which are positioned across the electric field-emitting surface 1311 (which in this embodiment may be two surfaces—the palm and back sides of the glove, or more generally around the entire exterior thereof) in a grid-like pattern. Alternatively, thin wire emitters may be woven directly into, or otherwise embedded in spaced relation to each other in, the cloth or other flexible substrate material. The “emitter head” portion 1301 may be made of a low-electric permittivity and non-conductive material, such as cloth, a synthetic fiber or a synthetic polymer, leather, or rubber, among others. The emitters 1330 may be secured to the surface 1311 of the cloth head portion 1301 by stitching, gluing, printing or any other form of secure affixing. The emitters 1330 also may be covered by an insulating layer of suitable permittivity so that accidental contact between individual emitters and resultant shorting may be prevented but the desired field delivered. Supply wire 1305 extends to the glove head portion 1301, connecting thereto at connection point 1307, where it splits-off into lead wires 1306 (shown as dotted lines), which connect with and bring electric current to each micro-strip electrode emitter 1330. Split-off lead wires 1306 may be woven within the material of the glove, placed in between two layers of cloth-like material or otherwise extend within the interior of the cloth material, to reach each emitter. In typical operation, the head component 1300 of FIG. 12 may be inserted over the hand of a user. In some embodiments, the interior portion of the glove head portion 1301 may have addition layers of non-conductive, low-electrical permittivity or shielding material to further protect the hand of a user. This configuration delivers an electric field or charge from each of the plurality of micro-strip electrode emitters 1330. The user may thusly grip or wipe various microbe-containing-surfaces (doorknobs, handles) while wearing the glove head/emitter portion 1301, so as to submit such target surface to contact (or near contact) with the electric field-emitting surface, and killing any microbes on such surface in the manner of irreversible permeabilization (electroporation) of the microbial cell wall, as discussed above.

In an alternative embodiment, the palm of the glove may be made of the conductive fabric described in connection with FIG. 12B. In this embodiment, the conductive fabric forming the glove palm is connected to the supply wire 1305, which may be connected at multiple points, so as to provide effective dispersion of the current flowing into the conductive fabric and thereby effectively disperse the electric field from the threads or filaments of the fabric. In one embodiment, the palm comprises an outer layer of the conductive fabric and an inner layer of a non-conductive material that spaces the user's hand from the conductive fabric, or a layer of non-conductive material and a second layer of the conductive fabric adjacent the user's hand and not connected to the supply wire 1305, to supply shielding.

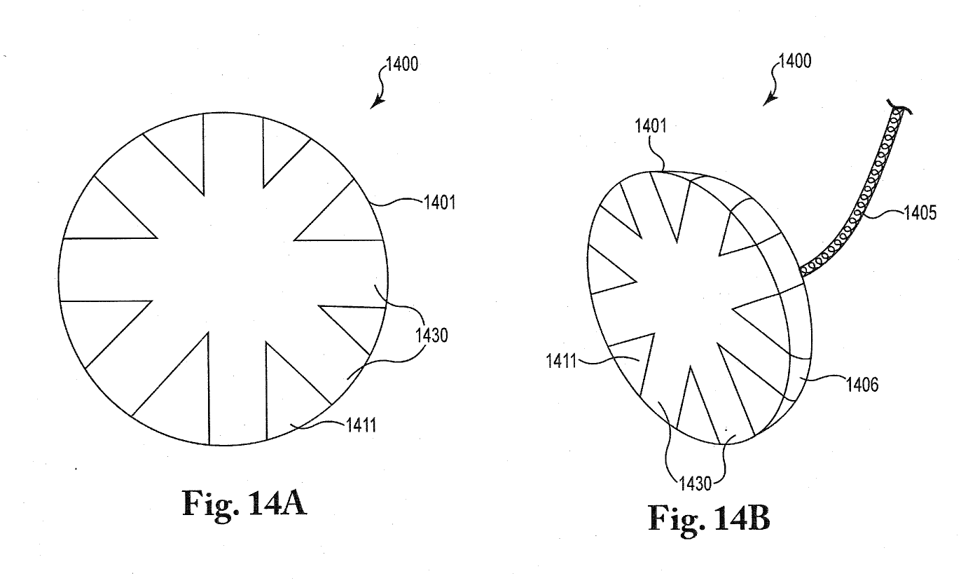

The embodiment of FIGS. 14A-B is an electric field-emitting head component 1400, of a generally circular shape, and having a plurality of long-strip emitters, such as copper strips, or other conductive metal foil strips which extend across the electric field-emitting surface 1411 and cross at a central point thereof. The wire 1405 splits-off to a plurality of wire leads 1406 extending about the exterior surface of the head portion 1401, which connect with and supply electric current to the plurality of long-strip emitters 1430 on the electric field-emitting surface 1411. This configuration delivers an electric field or charge from each of the plurality of long-strip emitters 1430.

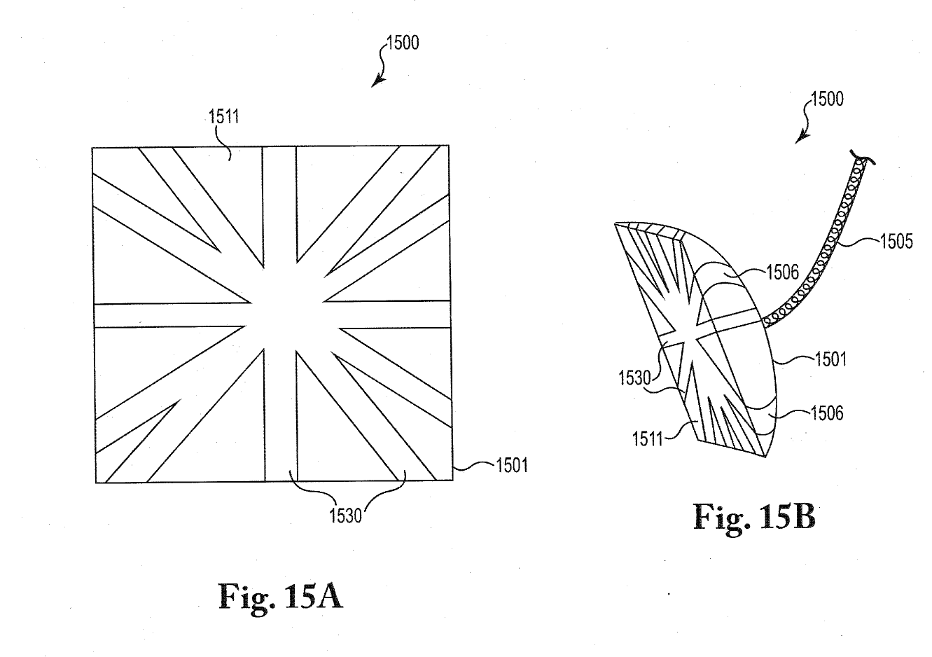

The embodiment of FIGS. 15A-B is an electric field-emitting head component 1500, of a generally square or rectangular shape, and having a plurality of long-strip emitters which extend across the electric field-emitting surface 1511 and cross at a central point thereof. The wire 1505 splits-off to a plurality of wire leads 1506 extending about the exterior surface of the head portion 1501, which connect with and supply electric current to the plurality of long-strip emitters 1530 on the electric field-emitting surface 1511. This configuration delivers an electric field or charge from each of the plurality of long-strip emitters 1530.

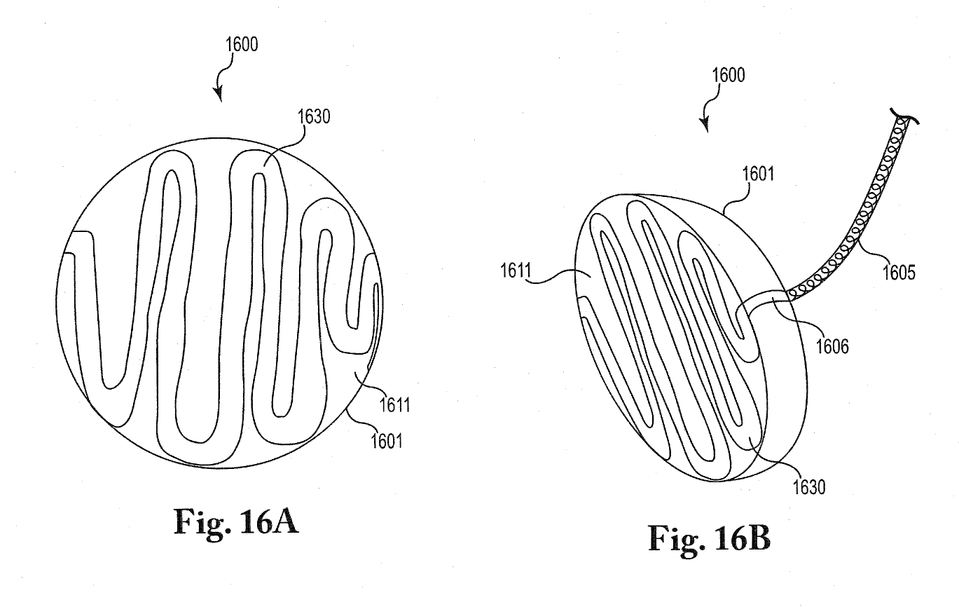

The embodiment of FIGS. 16A-B is an electric field-emitting head component 1600, of a generally circular shape, and having a single long-strip emitter extending across the electric field-emitting surface 1611, through a series of bends and curves, as shown best in FIG. 16A. The wire 1605 splits-off to a single wire lead 1606 running along the exterior surface of the head portion 1601, which connects with and supplies electric current to the long-strip emitter 1630 on the electric field-emitting surface 1611. This configuration delivers an electric field or charge from each of the plurality of long-strip emitters 1630.

As will be appreciated in accordance with these examples, electric-field emitting head components can be configured in many shapes or forms, and in many sizes, with various numbers or types of emitters associated therewith. Such additional configurations will be understood to be within the scope of this disclosure.

A benefit of the present device and method is that it destroys microbes on surfaces that are not suitable for application of an aqueous or other spray. For flat, smooth surfaces, such as tables or desks or food preparation surfaces, the embodiments shown above that have flat field emitting surfaces can be used to project a sufficient field to the target surface and its microbes. For rough or irregular surfaces, other flexible embodiments may be used to bring the electric field into intimate contact with rough or irregular shape of the target surface. Because air is relatively low permittivity in most circumstances, a large air gap between the field-emitting elements and the target surface reduces effectiveness; some surfaces are sufficiently rough that intimate contact between them and a typical emitter is not practical and may damage the emitter elements; conversely some emitters may damage target surfaces. Accordingly, the embodiments for these environments make use of several approaches. One strategy for rough or fragile target surfaces is to avoid the friction with protective projections or layers that can withstand the roughness and/or that reduce friction. Another strategy for surface irregularity that is more than microscopic is to fill the gap between field-emitting elements and the target surface with a field transport layer of a material having better permittivity than ambient air. In some cases, it is desirable to use the field transport layer combined with a protective, anti-friction layer. In further embodiments, a conductive fabric wiping cloth, which may allow direct contact between the field-emitting components and the target surface (or portions thereof), which may result in charge transfer to microbes, causing cell death at points of direct contact.

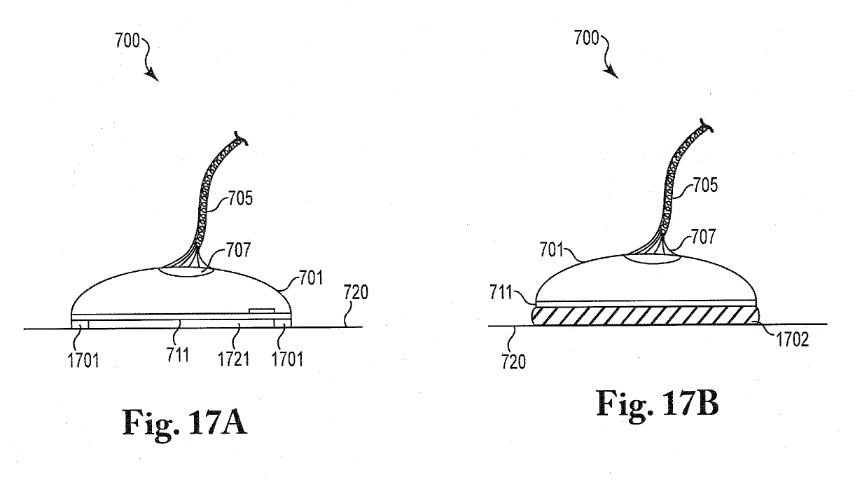

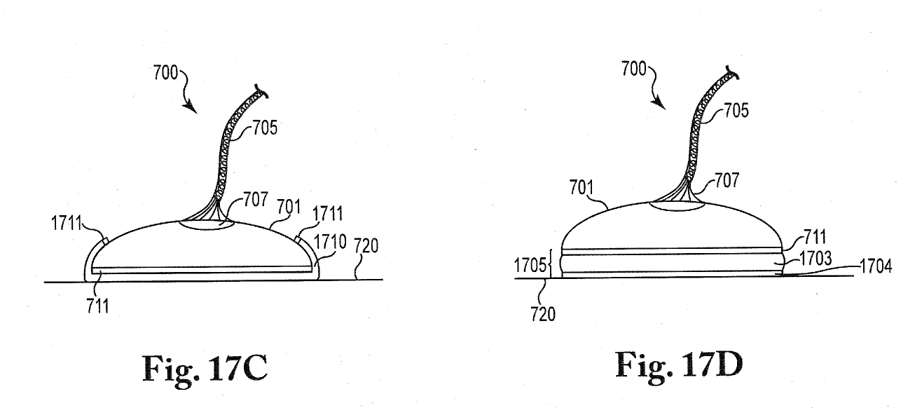

FIGS. 17A-17D depict example modifications to the electric field-emitting head component 700 to improve electric field projection, surface cleaning, rough surface damage prevention (for either the surface and cleaning head, on surfaces such as textured wall coverings, raw wood), and irregular surface contour conforming, wherein such modifications may be made to a generally planar electric-field emitting head component described above. As shown in FIG. 17A, the electric field-emitting head component 700 has been modified with a plurality of protective projections 1701. These may be positioned as projections from the plane of the electric field-emitting surface 711 at intervals about the perimeter of the electric field-emitting surface 711 of the head portion 701. Alternative embodiments may be configured with a single continuous protective rim 1701 about the entire perimeter of the electric field emitting surface 711. Other embodiments may use protective projections distributed also within the interior of the electric field-emitting surface 711. To reduce friction, reduce wear on surfaces cleaned and withstand wear caused by movement of protective projections 1701 over rough surfaces, these may be made of a hard, low friction resin, such as those placed on the bottom of furniture to allow it to slide. In one embodiment, the resin is selected from the group consisting of a nylon resin, acetal and other plastic or moldable materials. Dimensions and resilience of the protective projections are selected to allow close proximity of emitters and target surfaces that carry microbes, where direct target surface contact is avoided or reduced.

FIG. 17B depicts an alternative configuration of an electric field propagation-enhancement/surface damage prevention modification, which separates the electric field-emitting surface 711 and the microbe-containing surface 720 with a resilient contact layer 1702, such as chamois or other absorbent cloth-like or fiber-based material, or a sponge-like material. Its resilience permits it to deform slightly to accommodate an uneven surface. Additionally, the material may be absorbent and retain an electric field propagation enhancement substance having a high electrical permittivity, such as water. In some embodiments, achieving a permittivity of about 20 or 30 or more in this field transport layer 1702 is desirable. Such a layer 1702 may also perform a wiping function to remove dust and other light surface soil, totally separate from its function to enhance emitter effectiveness.

FIG. 17C depicts an alternative configuration of a surface wiping enhancement modification, which adds a replaceable surface-wiping layer 1710 to the electric field-emitting surface 711. The surface wiping layer 1710 may be a wiping cloth that removably attaches to the head portion 701 via attachment portions 1711. When applied across a surface, the surface cleaning layer may attract and retain dirt, soil, oils, liquids, or other compounds which it is desired to remove from a surface. The surface wiping layer 1710 is of a material that does not significantly impede the electric field generated from the electric field-emitting surface or may help deliver it; the layer 1710 may be relatively thin, for example less than 2.5 mm in thickness, or more preferably about 0.5-1.5 mm in thickness. In one embodiment, a conventional cotton or micro-fiber cleaning cloth fabric may be used as the field transport layer and wiping layer. Further, the layer 1710 may be made of a material with a high permittivity, or it may be made of a material that does not have a permittivity so low as to inhibit delivery of the electric field to the target surface. After use, the soiled surface wiping layer 1710 may be removed from the head portion 701 and disposed of. Alternatively, the surface wiping layer 1710 may be washable and reusable. In this embodiment, the device may thus by used both to destroy microbes on a surface, and at the same time to clean a surface of dust, dirt, oils, etc. Further alternatively, the surface wiping layer may be the conductive material described above in connection with FIG. 12B.

FIG. 17D depicts a further alternative configuration of an electric field propagation-enhancement/damage prevention modification, which also enables treatment of more irregular surfaces. Here the gap between the electric field-emitting surface 711 and the microbe-containing surface 720 is occupied by a resilient, electric field transport layer 1703 and an optional low friction layer 1704, forming a composite field transport layer 1705. The resilient, electric field transport layer 1703 may be made of a resilient material with high permittivity, or it may be an absorbent material (such as a chamois or sponge) with a high-permittivity medium absorbed therein, such as a liquid or gel with a permittivity of about 20 or 30 or higher. In one embodiment a colloid may be used, contained within a bag that is shaped to form a pancake-like layer and that permits the colloid to assume shapes that conform to irregular target surfaces, such as a doorknob, a faucet handle, a curved sink rim or table edge. In one embodiment a hydrocolloid may be used. The optional low friction layer 1704 may be a material that generally provides a low coefficient of static and dynamic friction when placed in contact with relatively smooth surfaces, such as tables, desktops, sinks, door handles, or other such surfaces. The composite 1705, when used in connection with the electric field-emitting head component 700, allows the device to conform to a variety of surfaces due to the deformability and resilience of the field transport layer 1703, allows the device to provide a strong electric field to the surface due to the favorable permittivity of the field transport layer 1703, and further allows the device to slide easily over surfaces due to the low friction layer 1704. Thus, the field transport material performs a function similar to a conductive fabric, as both help to extend the electric field into more intimate contact with the surface to be treated.

In an alternative embodiment, a surface cleaning layer 1710 (see FIG. 17C) may be used in place of, or in addition to low friction layer 1704, as described above with regard to FIG. 17C. In the above embodiments of FIGS. 17A-D, the electric field-emitting head component 700 may have emitters in the form of FIGS. 8A-8B, 10A-10B, 11A-11B. Further, as to FIGS. 10A-10B, 11A-11B, the substrate on which the emitters are formed may be of a material that flexes somewhat (i.e., may be bent in an arc), so as to enhance the ability of a head component with a deformable field transport layer to conform to a non-flat, non-uniform, or otherwise irregular target surface. Such conformity may serve to enhance the delivery of the electric field to the target surface.

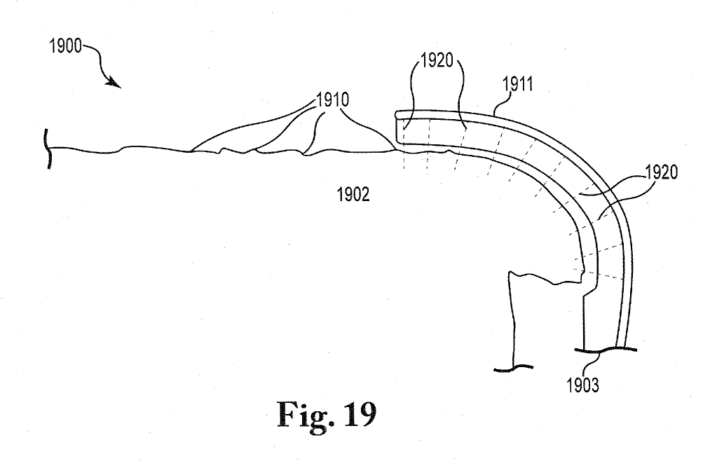

FIG. 19 shows schematically a surface 1900 at the edge of a counter or molding 1902 that is to targeted for microbe killing and is both irregular, by not being flat, and that has microscopic crevices 1910 that may harbor microbes. As can be seen, if the emitter 1911 is made of a flexible material and is mated with a resilient field transport layer 1903 (or a conductive extension of the emitter, such as the conductive fabric discussed above), it is able to conform to the curve of the molding, and the resilient field transport layer 1903 (or emitter extension) may deform enough at crevices that the field 1920 penetrates crevices to a sufficient extent that a field with strength effective to kill of microbes will extend into the crevice. If the molding has a porous surface, and microbes could enter more than just crevices 1910, the flexing of the emitter 1911 and the resilience of the field transport layer 1903 (or emitter extension) become even more important for delivery into a creviced or porous surface of a field effective to perform microbe killing. In some circumstances, the strength of the electric field will need to be increased to achieve field penetration of pores or deeper crevices.

### Controller and Control Board

It has been found that the effectiveness of the projected electric field in killing microbes on a surface may depend on the waveform, power level or other characteristics of the current driving the emitter. Thus, a suitable control and driver circuit is needed. In some embodiments, the supply wire 705 may be connected directly to a power source to supply a desired waveform and power level electric current to the emitters, thereby allowing a single, pre-determined form of electric field to be emitted. In preferred embodiments, however, the wire 705 may be electrically-connected to a control and supply board (and the control board being electrically connected to the power source), that allows the user to vary and select the waveform shape and power level and thus characteristics of the electric field to be emitted from the emitter. Such characteristics include the magnitude of the electric field, the intensity, waveform, and the pulse interval or frequency.

Generally speaking, the magnitude of an electric field, which is expressed in Newtons per Coulomb (N/C) or Volts per Meter (V/m), depends on the current and voltage supplied to the emitters, all other things being constant. Varying the current and voltage will vary the magnitude of the electric field, proportional to such variance.

The voltage waveform is simply a graphical representation of the electrical potential at the emitter over time. AC voltage waveforms may be regular sinusoidal waves, or they may be stepped, “saw-tooth,” or any other shape known to those in the art. In one embodiment, a pulse with a sharp rise time is used or a waveform with an irregular (not a pure sine wave) shape is used. Such pulses or waveforms are known from Fourier analysis to contain a mix of frequencies, including some higher than the fundamental frequency of a pulse train. A waveform generating component of the control board may serve to generate one or more of such waveforms. Waveforms are discussed in greater detail below.

The pulse interval simply refers to the duration and frequency at which the waveform and resulting electric field arc emitted (current is supplied to the emitters). As an example, the control board may be configured to supply current to the emitters in a repeating pattern of three pulses, each one a microsecond long, each one second apart from the next. Obviously, various pulse intervals may be selected, consistent with pulse duration. A pulse generating component of the control board may be controllable to generate such pulse intervals.

Referring now to an example configuration of a control board in accordance with the present disclosure, such a control board can include any suitable control circuit, which can be implemented in hardware, software, or a combination of both, for example, in order to generate a desired electric field magnitude, voltage waveform, and pulse interval. With particular regard to the waveform, the emitter can be supplied, or “driven” with any voltage waveform suitable to achieve the desired microbe de-activation level. The electrical characteristics of the driving voltage pattern will be based on the design of the apparatus and the method of application thereof. In one example, the driving voltage applied to the emitter has a frequency in the range of 15 kilohertz to 1500 kilohertz, or 40 kilohertz to 800 kilohertz, and a voltage of 50 Volts to 1000 Volts, or 50 Volts to 5000 Volts root-mean-square (rms). In some applications, the applied current can be very low, such as but not limited to the order of about 0.01, 0.05, 0.1, 0.15, 0.20 milliamps, or values in between, and yet still be sufficiently strong to destroy microbes. Using a low current may effectively prevent arcing between the emitter and the microbe containing surface. Alternatively, the current can be relatively high, such as but not limited to 0.20 milliamps-1000 milliamps, or even greater. In a preferred embodiment, the applied current can be about 1 to 6 mA, or about 2-5 mA, or about 3-4 mA.

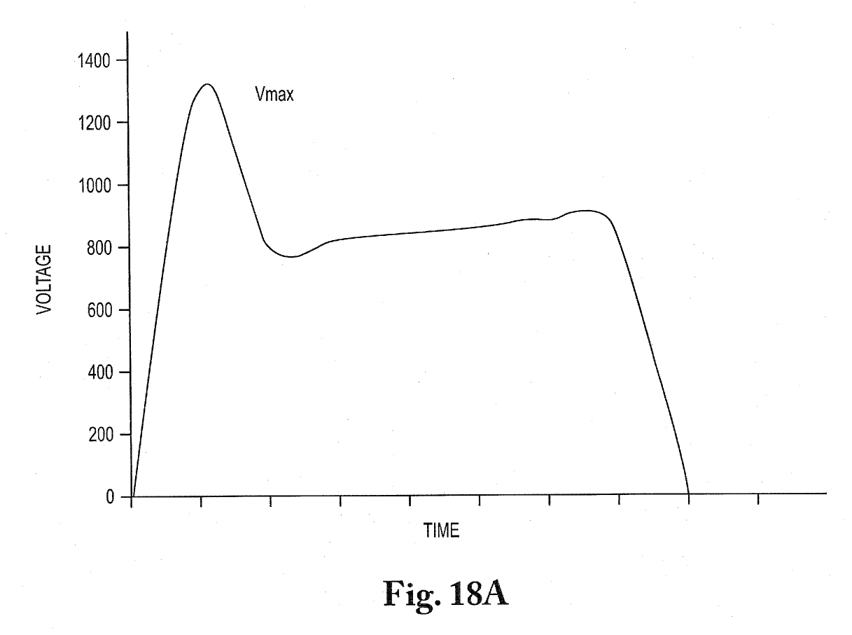

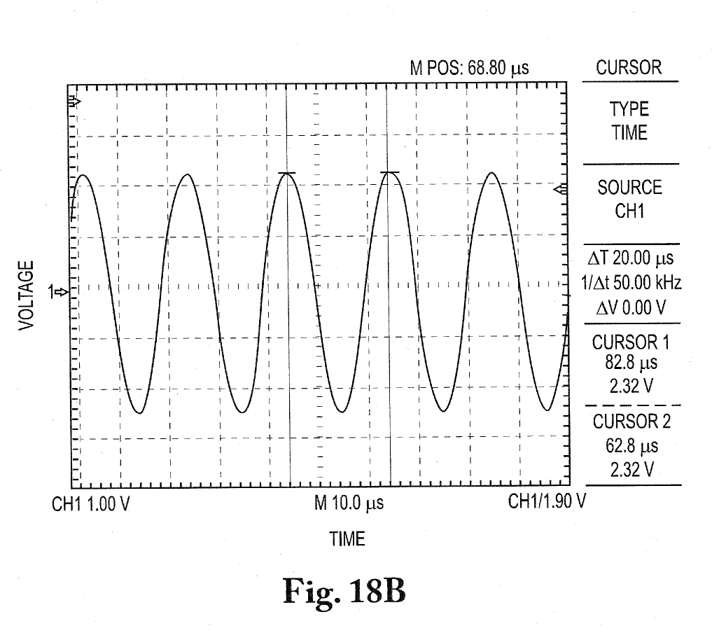

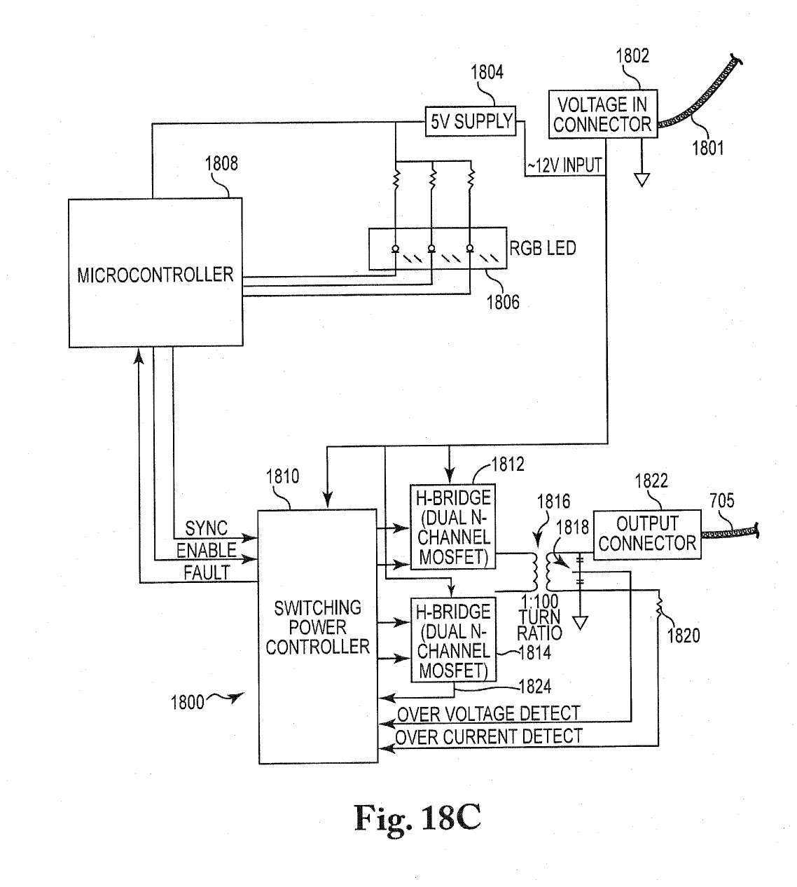

The voltage pattern can have a DC component, or be a pure. AC pattern. The voltage waveform can be any suitable type such as square, sinusoidal, triangular, saw-tooth, stepped (as shown in the example waveform of FIG. 18A), and/or arbitrary (from arbitrary pattern generator). In one example, the waveform sequentially changes between various waveforms. The positive (or alternatively negative) side of the voltage potential is applied to the emitter, and the potential of the microbe-containing surface being treated serves as the circuit ground (such as Earth ground), for example.

In addition, the waveforms and voltage levels may affect different microorganisms differently. So these parameters can be modified to enhance killing of particular microorganisms or can be varied during application to treat effectively a variety of different organisms. Examples of suitable voltages applied to the emitter include but are not limited to AC voltages in a range of 50 Vrms to 3000 Vrms, 700 Vrms to 2200 Vrms, or 1300 Vrms to 2000 Vrms. One particular embodiment applies a voltage of about 1500 to 1800 Vrms to the emitter. Examples of frequencies for the voltage that is applied to the emitter include but are not limited to those frequencies within a range of 10 KHz to 200 KHz, 20 KHz to 100 KHz, 25 KHz to 75 KHz, 30 KHz to 65 KHz, or about 45 Khz to about 55 KHz. One particular embodiment applies the pulse at a fundamental frequency at about 30 KHz to the emitter.

FIG. 18A is a waveform diagram illustrating the voltage pattern applied to the emitter in one particular example. In this example, the shape of the waveform is a stepped square wave. FIG. 18B is a waveform diagram illustrating the voltage pattern applied to the emitter in another example. In this example, the shape of the waveform is roughly a sine-wave, with approximately 20 micro-seconds from peak to peak of each wave (indicating approximately 50 kHz). However, the waveform can have other shapes, such as a modified sine wave, a saw-tooth wave, or other waveform. The frequencies mentioned above are nominal, and correspond to the fundamental frequency of the waveform, which in the case of anything other than a pure sine wave will also contain other frequencies that are part of the particular waveform.

In some embodiments, the frequency may remain substantially constant as the apparatus is used in treating a microbe-containing surface. In another example, the frequency varies over a predefined range while the apparatus is in operation. For example, the control circuit that drives emitter can sweep the frequency within a range between a lower frequency boundary and an upper frequency boundary, such as between 20 KHz to 200 KHz, 25 KHz to 100 KHz, 30 KHz to 65 KHz, or about 45 Khz to about 55 KHz. In another example, the control circuit ramps the frequency from the low frequency boundary to the high frequency boundary (and/or from the high frequency boundary to the low frequency boundary) over a time period of 0.1 second to 15 seconds. Other ramp frequency ranges can also be used, and the respective ramp-up and ramp-down periods can be the same or different from one another. Since different microbes may be susceptible to irreversible electroporation at different frequencies, the killing effect of the applied voltage is swept between different frequencies to potentially increase effectiveness on different microorganisms. For example, sweeping the frequency might be effective in applying the potential at different resonant frequencies of different microorganisms. In one particular example, the frequency is swept between 30 KHz and 70 KHz with a saw-tooth waveform. Other waveforms can also be used.

FIG. 18C is a block diagram illustrating an example of a control board circuit 1800 for providing a voltage potential to an emitter. Circuit 1800 may include a voltage input connector 1802, a voltage regulator 1804, a tri-color LED 1806, microcontroller 1808, switching power controller 1810, H-bridge circuits 1812 and 1814, transformer 1816, voltage divider 1818, sense resistor 1820 and output connector 1822, in addition to filler material located across the board (not shown) to protect the board from moisture. Input connector 1802 may receive the supply current and voltage through wire 1801 from the power source (not shown), and may supply the voltage to voltage regulator 1804, switching power controller 1810 and H-bridge circuits 1812 and 1814. In a particular example, voltage regulator 1804 may provide a 5 Volt output voltage for powering the various electrical components within the control circuit 1800, such as microcontroller 1808, LED 1808 and Switching power controller 1810. Any suitable voltage regulator can be used, such as an LM7805 regulator from Fairchild Semiconductor Corporation.

In this embodiment microcontroller 1808 may have three main functions; providing a clock signal (SYNC) and an enable signal (ENABLE) to switching power regulator 1810, monitoring for fault conditions (indicating that the control board is not functioning properly, i.e., not providing electric current to the emitter), and providing a user an indication of a fault condition through LED 1806. In one example, microcontroller 1808 may include an ATtiny24 QPN Microcontroller available from ATMEL Corporation. Other controllers can be used in alternative embodiments. The clock signal SYNC may provide a reference frequency for switching power controller 1810. Enable signal ENABLE, when active, may enable (or turn on) switching power controller 1810. Normally, microcontroller 1808 sets ENABLE to an active state and monitors the FAULT signal for a fault condition. When no fault condition is present, microcontroller 1808 may selectively turn on one or more colors of the tri-color LED 1106. In one example, LED 25 1806 is a tri-color red, green, blue LED. However, multiple, separate LEDs can be used in alternative embodiments. Further, other types of indicators can be used in addition or in replace of LED 1806, such as any visual, audible or tactile indicator. When controller 1810 indicates a fault condition by activating the signal FAULT, microcontroller 1808 may selectively pulse the ENABLE signal to an inactive state and then returns it to the active state to reset switching power controller 1810. This may be indicated by illuminating the blue LED. If the fault condition clears, microcontroller continues to illuminate the blue LED. If the fault condition remains active, then microcontroller turns off the blue LED and illuminates a red LED. The green LED is not used in this example, but could be used in alternative embodiments. Other user indication patterns can, be used in alternative embodiments.

In one example, switching power controller 1810 may include a TPS68000 CCFL Phase Shift Full Bridge CCFL Controller available from Texas Instruments. However, other types of controllers can be used in alternative embodiments. Based on the SYNC signal, switching power controller 1810 may provide gate control signals to the gates of switching transistors within the H-bridge circuits 1812 and 1814. In one example, H-bridge circuits 1812 and 1814 may each include an FDC6561AN Dual N-Channel Logic Level MOSFET (although other circuits can be used), which are connected together to form an H-bridge inverter that drives the primary side of transformer 1816 with the desired voltage pattern, such as that shown in FIG. 18A. Transformer 1816 may have about a 1:50 turn ration, about a 1:100 turn ratio, about a 1:200, or about a 1:500 turn ration, or any ratios therebetween effective to achieve a desired output voltage. The transformer 1816 may step the drive voltage from about 10V-13V peak-to-peak up to about 1000V-1300 V peak-to-peak (about 600 V rms), for example. The output drive voltage may be applied to the emitter through output connector 1822, which in turn is connected to wire 705.

Voltage divider 1818 may include a pair of capacitors that are connected in series between the primary side of the transformer and ground to develop a voltage that is fed back to switching power controller 1810 and represents the voltage developed on the secondary side of the transformer. This voltage level may be used to detect an over-voltage condition. If the feedback voltage exceeds a given threshold, switching power controller 1810 may activate fault signal FAULT. Sense resistor 1820 may be connected between the primary side of the transformer and ground to develop a further feedback voltage that is fed back to switching power controller 1810 and represents the current flowing through the secondary side of the transformer. This voltage level may be used to detect an over-current condition. If the feedback voltage exceeds a given threshold, switching power controller 1810 may activate fault signal FAULT, indicating a fault in the transformer. In addition, the source of the bottom transistor in one leg of the H-bridge may be fed back to switching power controller 1810, as shown by arrow 1824.

This feedback line can be monitored to measure the current in the primary side of the transformer, which can represent the current delivered to the load through the emitter. Again, this current can be compared against a high and/or a low threshold level. The result of the comparison can be used to set the state of fault signal FAULT. Alternatively, the voltage level may be regulated based on sensing directly or indirectly a field strength that is being delivered. The voltage and resulting field output may then be adjusted to deliver a stronger or weaker field as may be called for by various target surfaces or microbe destruction goals.

In some embodiments, the control board may be further configured with a protective or fuse-like circuitry or over-current control to detect a rapid or otherwise unusual increase in current. In response, the control hoard may cut power to the emitter, or at least significantly reduce power, to prevent arcing between the emitter and the surface, and also to prevent damage to the control board components. This capability may be particularly useful where a conductive cloth is used as an emitter and the cloth may momentarily contact some highly conductive material. It is desirable both to protect circuit components and to prevent any significant arcing. A fast-reacting current limiting circuit may provide this facility and simply cut or limit the current for a period rather than tripping a fuse that must be reset.

A further reason for over-current control is that the present device operates under near-field conditions. In analyzing the effect of electric fields, one distinguishes between the “far field”, which generally extends from about two wavelengths distance from the emitter to infinity and the “near field”, which is inside about one wavelength's distance from the emitter. In the near field, there are strong inductive and reactive effects from the currents and charges on the emitter. Because the close contact of emitters and surfaces treated contemplated by the embodiments shown herein, it is believed that the behavior of the emitter and fields will be near-field behavior. Absorption of radiated power in a near-field zone has effects which feed back to the emitter, increasing the load on the circuit driving the emitter by decreasing the impedance the driver circuit sees.

Referring again to FIG. 19, it should be noted an emitter 1911 made of a flexible material and mated with a resilient field transport layer 1903 (or a conductive extension of the emitter, such as the conductive fabric discussed above) conforms to the larger shape of the surface to be treated. In addition, the resilient field transport layer 1903 (or emitter extension) may deform enough into more microscopic surface irregularities that the field 1920 penetrates crevices and pores to a sufficient extent that a field with strength effective to kill of microbes will extend into the crevices and pores. Thus, the structure brings the electric field into intimate contact with the surface to be treated. This enables a method for killing microbes by providing an electrically conductive emitter for emitting an electric field for killing microbes in contact with or in close proximity to the emitter; and providing a control circuit for electrical connection to the emitter to deliver a current with an AC pulse waveform having a fundamental frequency in the range of 10 KHz to 200 Hz, said control circuit being activated to deliver the current for a defined interval, and causing the emitter to emit an electric field sufficient to cause electroporation of microbes in contact with or in close proximity to the emitter, said current being controlled to a level that limits arcing from the emitter to adjacent objects. Alternatives for this method include providing an emitter selected to conform to a surface to be cleaned, providing an emitter that is conformable into intimate contact with a portion of a surface to be cleaned or providing an emitter consisting of an array of separate emitters on a substrate conformable into intimate contact with a portion of a surface to be treated.

### Power Source

A power source (not shown in FIG. 18C) is provided to supply current and voltage to the emitter on the electric field-emitting component. The power source may be connected the control board which manipulates the current, voltage, frequency, etc. of the power supplied therefrom. In a one embodiment, to provide a portable device, the power source is a battery pack having a plurality of batteries therein, connected in series to one another and in turn connected to a control board, for example, control board 1800 at a voltage input connector 1802.

In alternative embodiments, the power source may be another form of battery or battery pack, a 110 volt outlet, a 220 volt outlet, a generator, a solar panel, a fuel cell, or any other source capable of generating voltage and current.

### EXAMPLE NO. 1

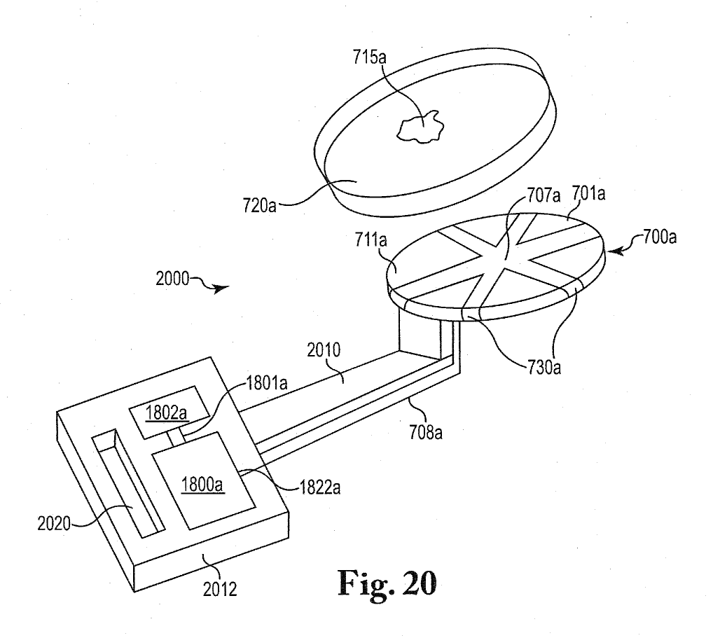

A configuration of an apparatus 2000 in accordance with the present disclosure is generally depicted as FIG. 20. Referring to FIG. 20, the electric field-emitting head component 700a is shown in a circular configuration, with the head portion 701a having a plurality of long-strip emitters in the manner of FIGS. 14A-B. The component 700a is approximately 8 cm-10 cm in diameter, with the long strip emitters being approximately 1 cm in width. A user handle 2012 may extend from the head 700a and have a finger aperture 2020. Emitters 730a extend from the electric field emitting surface 711a about the exterior of the head portion 701a. Split-off wire leads (not shown) extend from the wire 705a at the connection point 707a to supply electric current to each of the plurality of long-strip emitters. Wire 705a delivers current and voltage at a particular magnitude, waveform, and pulse interval as generated by the control board 1800a mounted in the user handle 2012, to which wire 705a is connected at output connector 1822a. Control board 1800a, as discussed above, has waveform generating components and pulse interval generating components thereon (not separately indicated). Control board 1800a, in turn, receives electric power through wires 1801a from power source 1802a, which may be a battery pack as discussed above. An example microbe-containing surface 720a, in the form of a Petri dish, is shown in the background. The microbe-containing surface 720a includes standard testing microbes which behave similarly to staphylococcus aureus, escherichia-coli, myobacterium, and spores, among others, under the testing conditions described below, but are less virulent than those microbes, thereby allowing the testing to be conducted without the need to guard against environmental contamination.

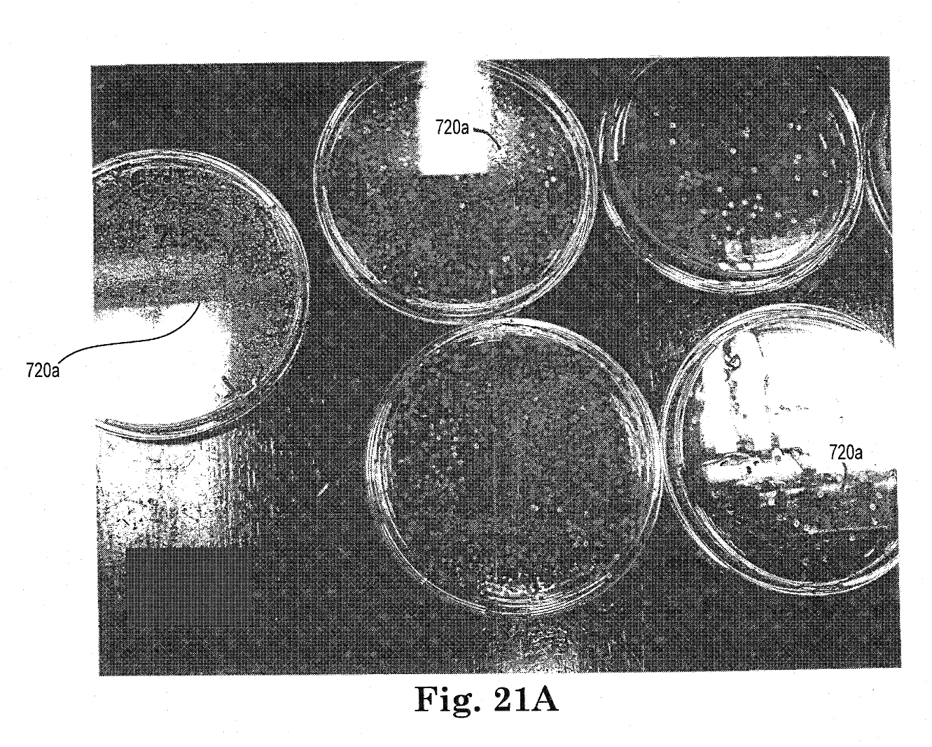

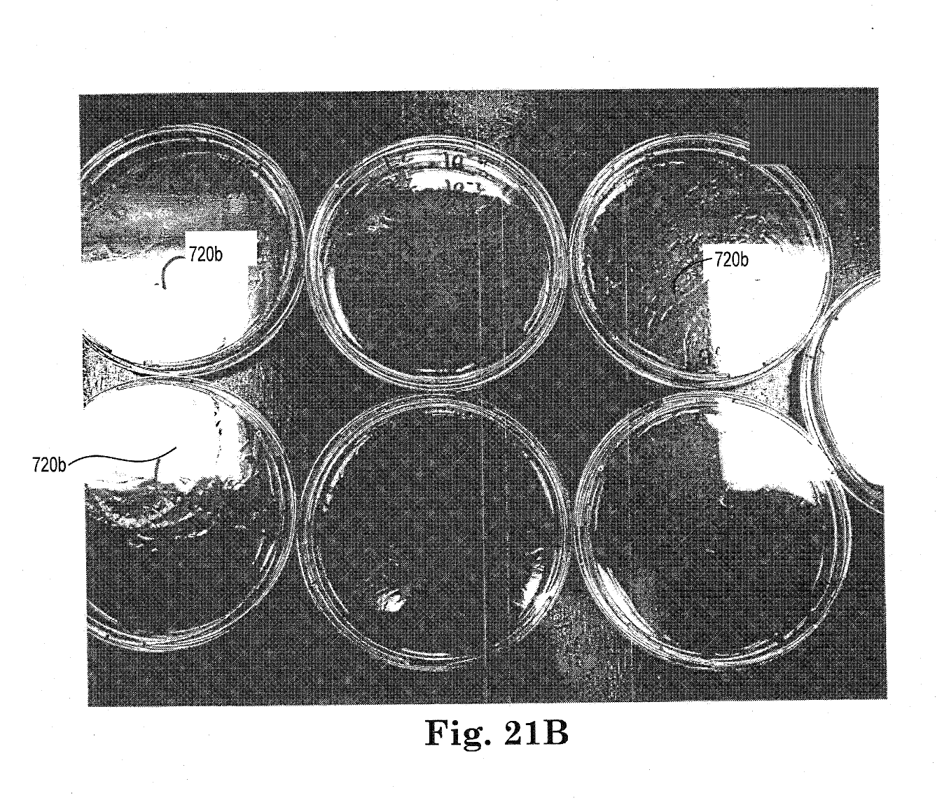

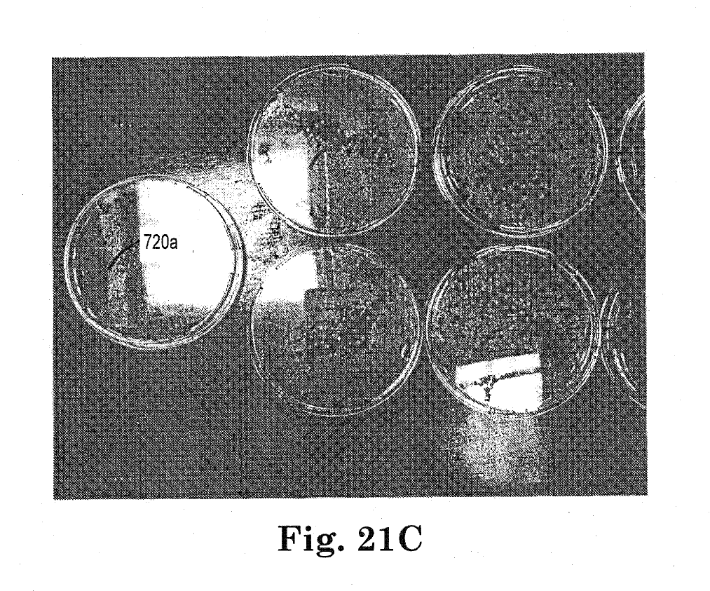

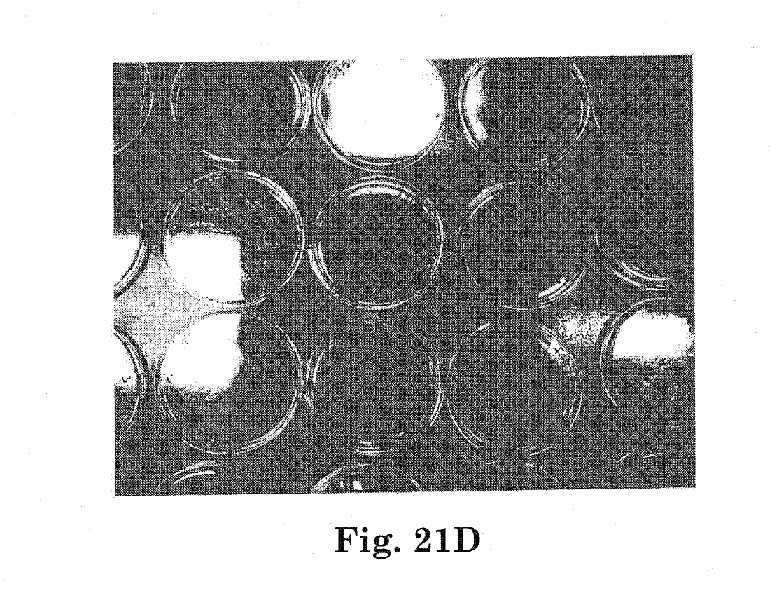

The efficacy of the apparatus configuration 2000 was tested on a plurality of microbe-containing surfaces 720a, in the form of Petri dishes having diameters slightly larger than the diameter of the head component 700a, as shown in FIG. 21A. This test was generally conducted under the standards set forth by AOAC International, and the Environmental Protection Agency. Each Petri dish had colonies of bacteria 715a living thereon. A small amount of water, less than one teaspoon, was placed on the surface of each Petri dish, thereby forming a thin layer of water above the microbe-containing surface. The apparatus 2000 was then applied to the Petri dish, wherein the electric field-emitting head component was brought into contact with the thin water layer. An electric field was applied in 1, 2, 3, 4, 5, or more approximately ½-second pulses, with 1500-1800 Vrms, 11-12 mA, and approximately 45-55 kHz.

As shown in FIG. 21B, the surfaces 720b (Petri dishes) no longer have visible colonies of bacteria 715a living thereon, thus demonstrating the effectiveness of the experimental apparatus 2000. Without binding or limiting the present invention to any particular theory of operation, it is hypothesized that the exposure of the bacteria to the electric field emitted from the electric field emitting surface 711a of the apparatus 2000 caused irreversible permeabilization of the cell membrane of bacteria 715a formerly present on the 720a (FIG. 21A), as discussed in detail above, thereby ultimately causing cell death. These results indicate that the apparatus effectively operates as a microbe-destroying device.

### EXAMPLE NO. 2

In another example, a device as described above with regard to Example No. 1 was used to test the efficacy of the apparatus configuration 2000 on a plurality of microbe-containing surfaces 720a, in the form of Petri dishes having diameters slightly larger than the diameter of the head component 700a, as shown in FIG. 21C. This test was also generally conducted under the standards set forth by AOAC International, and the Environmental Protection Agency. Each Petri dish had colonies of bacteria 715a living thereon. In this example, no water was placed on the surface of the Petri dishes. Only a small layer of air (e.g., less than 2 mm, in some areas less than 1 min) separated the electric field emitting surface 711a from the surfaces 720a. An electric field was applied in 1, 2, 3, 4, 5 or more pulses of 1-second duration, with 1500-1800 Vrms, 11-12 mA, and approximately 45-55 kHz.

As shown in FIG. 21D, the surfaces 720b (Petri dishes) no longer have visible colonies of bacteria 715a living thereon, thus demonstrating the effectiveness of the experimental apparatus 2000. Without binding or limiting the present invention to any particular theory of operation, it is hypothesized that the exposure of the bacteria to the electric field emitted from the electric field emitting surface 711a of the apparatus 2000 caused irreversible permeabilization of the cell membrane of bacteria 715a formerly present on the 720a (FIG. 21C), as discussed in detail above, thereby ultimately causing cell death. These results indicate that the apparatus effectively operates as a microbe-destroying device without any liquid medium between the electric field-emitting surface 711a and the microbe-containing surface 720a.

### EXAMPLE NO. 3

In another example, a device with a rectangular head as described above with FIG. 12B was used to test the efficacy of the apparatus using a conductive fabric as the primary emitter surface on a microbe-containing surfaces 720a, in the form of a glass Pyrex pan (9×13) contaminated with bacteria. The test with the fabric was performed by: (a) applying power to the emitter head; (b) moving the emitter head back/forth in a “mopping motion” across the pan bottom area where the bacteria were located; and (3) using microbiology methods to test the efficacy of the device (i.e., testing the pan and conductive fabric to see if any bacteria survived). An electric field was applied during the “mopping motion”, with 1, 2, 3, 4, 5 or more pulses of 1-second duration, with 1000-1800 Vrms, 4-9.5 mA, and approximately 30 kHz. After this “mopping motion” no significant bacteria survival was detected

### Hand-Held Devices

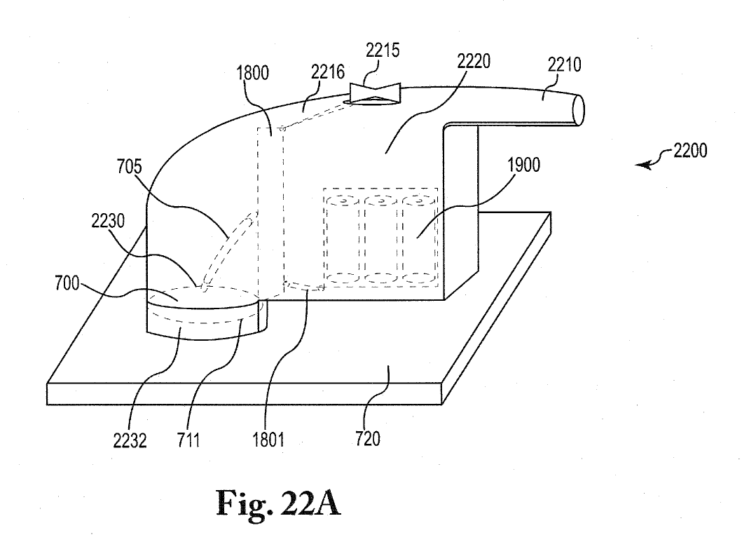

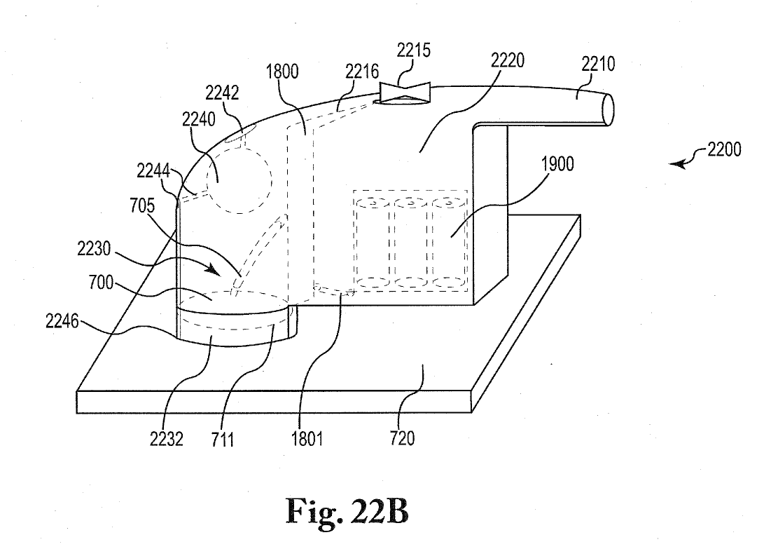

Some embodiments of the present disclosure may be configured in the form of a hand-held apparatus. As shown in FIG. 22A, a hand-held apparatus for disinfecting microbe-containing surfaces 2200 includes a handle portion 2210, a body portion 2220, and a head portion 2230. The handle portion 2210 may be connected to the body portion 2220 at an end thereof. The handle portion 2210 may be designed so as to allow a user to easily and ergonomically grip and maneuver the apparatus. The head portion 2230 may generally extend from the body portion 2220 at an end opposite the handle portion 2210, as shown in the example apparatus of FIG. 22A.

The body portion 2220 defines an interior volume. In alternative embodiments, the handle portion may also define an interior volume. Within such volume may be included the power source, which may be in the form of a battery pack 1900, the control board 1800, and also the wire 1801 operably connecting such components to one another (all shown in dotted outline). The head portion 2230 also defines an interior volume. Within such volume may be included the electric field-emitting head component 700, and the wire 705 which operably connects the component 700 to the control board 1800 (again, shown in dotted outline). The head portion 2230 has an opening 2232 on the under surface thereof to expose the electric field-emitting surface 711, and the emitters thereon (not shown) to a microbe-containing surface 720.

In order that a user may operate the apparatus, a user control component 2215 may be provided, positioned on an exterior surface of the body portion 2220 and proximate the handle portion 2210. The user control component 2215 may be connected to the control board 1800 by means of a wire 2216 positioned within the interior volume of the body portion 2220 (shown in dotted outline). The user control component 2215, in one embodiment, may be a switch which only allows the user to turn the apparatus off and on—that is, the user only controls whether the apparatus is operating, not any of its functional parameters, e.g., electric field magnitude, voltage waveform, and pulse interval. In a preferred embodiment, however, the user control component includes the switch as described above, and it also includes one or more buttons, dials, knobs, etc., which allow the user to adjust the functional parameters of the apparatus, including the electric field magnitude, voltage waveform, pulse interval, and other parameters as described in greater detail above. The user may manipulate such buttons, dials, knobs, switches, etc., causing the wire 2216 to transmit a signal, which may be in digital or analog form, to the control board 1800. Such signal causes an adjustment to the components of the control board, for example the voltage waveform generating component and/or the pulse interval generating component, to cause the apparatus to operate in accordance with the user selected parameters.

FIG. 22b depicts an alternative embodiment of the hand-held apparatus 2200 of FIG. 22B. In this embodiment, also included in the interior volume of the head portion 2240 is a reservoir 2240, configured to hold a volume of water or other liquid that may be delivered in the form of a mist to the target surface. In this embodiment, the user may add water, or any other liquid, useful for conventional wiping, through the opening 2242, thus filling the reservoir 2240. The reservoir 2240 is connected through a series of tubes 2244 within the interior volume of the head portion 2230. The tube channels the liquid to a dispensing component 2246 positioned adjacent to the opening 2232. In this manner, the apparatus may dispense a mist which wets the microbe-containing surface 720 as the user maneuvers the apparatus across a target surface. The mist prepares the surface for a conventional wiping with a separate fabric cloth or paper towel when needed to remove certain substances, such as the sticky residue of a spilled beverage. This misting and wiping could be done before or after treatment with the electric field. The dispensing of such mist may be controlled by the user control component 2215, or it may be controlled automatically by the control board 1800 without allowing for adjustability by the user.

A variation of the embodiment of FIG. 22B may include a heating element, powered by the power source, and position proximate the tubes 2244 or the reservoir 2240. In this manner, water contained within the reservoir may be heated to provide steam or warned, humidified air through the dispensing component 2246 (as the medium 710).

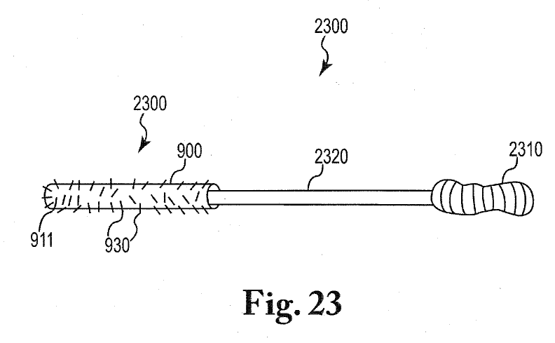

In a further embodiment of a hand-held apparatus 2300 in accordance with the present disclosure, as shown in FIG. 23, the apparatus may be configured in the form of a wand with a brush head. This embodiment of a hand-held apparatus generally functions as described above with regard to FIGS. 22A, with the following differences: In this embodiment, the handle portion 2310 may generally be more rounded, to allow the user to easily manipulate the wand shaped apparatus 2300, in the manner of a dusting wand, for example. The body portion 2320 may be configured in the form of a long tube, or wand, which in some variations may be a telescoping wand. The head portion 2330 may be configured as a “brush” electric field-emitting head component 900 (having a plurality of wire emitters extending from the surface thereof), in the manner of FIG. 9, discussed above. It is envisioned that this embodiment may be employed by a user desirous of disinfecting “hard to reach” surfaces 720, such as tops of shelves, cabinets, or any other surface 720 which may be difficult to disinfect with the previously described apparatuses.

To make the devices shown above, more flexible, the emitter components can be detachable. Thus, a controller that has a detachable connector for its output current may be connected to and used with any of the head components discussed above. Here, the heads would be interchangeable and connectable to the controller, to be adapted to varying surfaces to be treated for microbe killing.

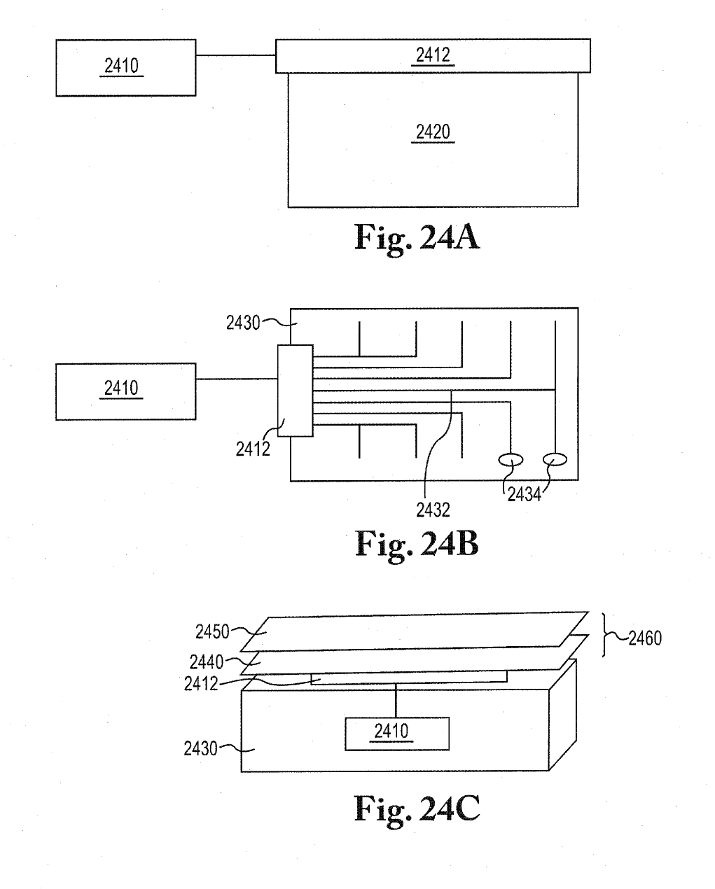

Use of a controller with a detachable connector for its output current opens up other possibilities, as shown in embodiments of the present disclosure depicted in FIG. 24A-24C. In these embodiments, the electric-field emitting component is an object separable from the controller 2410 that houses the control board and the power source with a larger area for which microbe-killing treatment is desired. In place of a permanently attached emitter component, a detachable connection means 2412 with a conductor attached to the output of the control board is provided to allow the controller to operably connect with an electric-field emitting component that is not a tool of the kind used to approach a surface to be treated; rather the electric-field emitting component is itself an object that has another function or is part of an object having another function and has a large area to which it is desirable to apply a field for killing microbes. The range of objects to which a field may be applied varies widely; thus the detachable connection means 2412 varies to facilitate attachment to one or more different types of emitter components.

To address larger areas for killing microbes, as seen in FIG. 24A, the controller 2410 may have a linear emitter connector 2412 that detachably connects to deliver current to a flat sheet of material 2420 to be treated for microbes. Said material is capable of functioning as an emitter so as to deliver the controlled electric field caused by the delivered current essentially simultaneously to all points on the material 2420. In this embodiment, the surface or element 2420 to be cleaned and which becomes an emitter component may be made from a conductive material that permits it to function as an emitter when electrically connected to the output of the controller 2410. This element 2420 can then expose the microbes on or in its surfaces and any other surfaces in close proximity to it to the fields that it emits.

In this embodiment, because the emitter component 2420 is detachable from the controller 2410, the emitter component 2420 can be a permanent fixture or other object that may need to stay mostly in one place, or an object that is difficult to effectively traverse completely with a head component as described above with respect to the embodiments of FIGS. 7 through 23. Examples of detachable emitter components include items with a large working surface where the killing of microbes on or within the surface of the item is desired. In one example, the detachable component 2420 is a table or the surface layer of a table, such as a patient examination or operating table in a health care facility. The table surface may be itself conductive, such as being made of a metal, or it may include emitting components integrally connected therewith, such as a wire mesh integrated on or in a non-conductive material, such as a plastic or a synthetic quartz countertop type material.

In another example, the detachable emitter component 2420 is a cutting board or other food preparation surface that is constructed with a conductive layer to which the controller 2410 may be electrically connected. The electrical connection 2412 may be by a single clip or clamp contact at one location on an edge, or, for a larger emitter component 2420, by an extended clip 2412 that makes continuous contact along an extended portion of an edge (see FIG. 24A) or by multiple electrical connectors that make contact at several distributed points along an edge or multiple edges. In a further example, the detachable emitter component 2420 is a flexible sheet material, such as a covering or cover layer for a table or other working surface, or a curtain, such as a patient separating curtain in a health care facility. Here, the curtain itself may be conductive (e.g., made of a conductive material such as discussed in connection with FIG. 12B), or may include emitting components, such as wires integrally contained therein. Other examples of detachable emitting components are possible.

For a larger emitter component 2420, a significant consideration is to deliver the electrical field relatively uniformly to essentially the entire emitter component, so that the microbe killing effect covers the entire component essentially simultaneously when controller 2410 delivers current. In addition to an extended edge connector as shown in FIG. 24A, FIG. 24B shows a connector 2412 attached to an emitter field distribution network 2432 to help create an effective field at all points of emitter component 2430. Such a network 2432 may comprise emitting wires or printed conductive paths that provide a field at all points along their length or may include wires or printed conductive paths that deliver current to area connectors 2434 (only two examples are shown for simplicity) that are focused on producing a field primarily in a defined areas, such as a 2×2 inch, or 4×4 inch area. This permits an extension of the operating principles described above for smaller emitters to provide microbe filling fields over larger areas.

Turning now to FIG. 24C, the controller 2410 and the connector 2412 may be part of a larger fixture 2430, such as an elevated working surface for food preparation or other activities where microbes are undesired. Emitter component 2440 is part of composite layer (shown exploded at 2460 for purposes of explanation) that forms the working surface. The outermost layer can be a plastic sheet or film selected for appropriateness to the work to be performed on it, and the emitter component 2440 may be bonded to it. Thus, the emitter component 2440 can be selected for its ability to project the desired electrical field, rather than appropriateness for contact with the work. If the composite is a disposable or is from time to time replaces, the connector 2412 is made easily detachable. In the case where the composite 2460 is a more or less permanent surface for fixture 2430, then detachability is not important.

In any of the embodiments, connector 2412 may be an interlocking connector that securely electrically connects to the emitter components 2420, 2430, 2440 while the apparatus is in operation. It may be detached by simple manipulation of the interlocking connector. Suitable configurations of such means 2412 may include connectors with a linear or multiple-spaced copper or other conductor contacts, such that the voltage and current introduced to the emitter may be introduced along a line or at multiple points on the emitter component, rather than at a single point.

In use, the controller 2410 supplies an electric current to the detachable connector 2412. Current flows to the emitter components 2420, 2430, 2440, and, by virtue of its own conductivity, or the conductivity of the emitting components included, an electric field or flow of charge is emitted from the detachable head in the same manner as discussed with regard to the above embodiments. In this manner, large, difficult to sanitize objects become more easily cleaned by eliminating the need to move the smaller heads of the above-disclosed apparatus over all portions thereof. Rather, microbe killing electric fields and current are supplied to the entire head 2401 (the entire detachable emitter) at once by virtue of the connection to the body portion and the conductivity of the head or the emitting components embedded therein.

Although the present disclosure has been described with respect to various embodiments, persons skilled in the art will recognize that changes may be made in form and in detail without departing from the spirit and scope of the present disclosure.

As used herein, the terms “front,” “back,” and/or other terms indicative of direction are used herein for convenience and to depict relational positions and/or directions between the parts of the embodiments. It will be appreciated that certain embodiments, or portions thereof, can also be oriented in other positions.

In addition, the term “about” should generally be understood to refer to both the corresponding number and a range of numbers. In addition, all numerical ranges herein should be understood to include each whole integer within the range. While an illustrative embodiment of the invention has been disclosed herein, it will be appreciated that numerous modifications and other embodiments may be devised by those skilled in the art. Therefore, it will be understood that the appended claims are intended to cover all such modifications and embodiments that come within the spirit and scope of the present invention.

What is claimed is:

1. An apparatus for emitting a controlled electric field for selective killing of microbes, comprising:
a control circuit, connectable to a power source, and comprising a current waveform generating component, wherein the control circuit receives an input electric current from the power source, and wherein the current waveform generating component transforms the input electric current into an output electric current with a predetermined waveform; and
an electric field emitting component, for receiving output electric current from the control circuit, comprising at least one emitter for emitting an electric field, wherein the pulse interval generating component transmits the output electric current from the control circuit to the emitter, thereby causing a controlled electric field to be emitted from the emitter with a predetermined waveform, sufficient to cause irreversible permeabilization of a cell membrane of microbes on the electric field emitting component or on a microbe-containing surface proximate to the electric field emitting component.

2. A hand-held apparatus for killing microorganisms on a microbe-containing surface, comprising:
a body portion;
a user control component positioned on an exterior surface of the apparatus
a control circuit, connected to the user control component; and
a head portion, extending from the body portion, connected to the control circuit, and comprising an emitter on an electric field-emitting surface thereof, wherein actuation of the user control component causes the control circuit to transmit an electric current to the emitter, thereby causing the emitter to emit an electric field from the electric field-emitting surface, sufficient to cause irreversible permeabilization of a cell membrane of microbes on a microbe-containing surface proximate to the head component.

3. A method for killing microorganisms on a microbe-containing surface using a controlled electric field, comprising:
providing a head component comprising an array of emitters on an electric field-emitting surface thereof;
providing a control circuit comprising an actuator, electrically connected to the head component, and configured such that when the actuator is actuated, the control circuit transmits an electric current having a voltage waveform to the emitters at a pulse interval;
positioning the head component such that the electric field-emitting surface is facing toward and positioned proximate to a microbe-containing surface; and
actuating the actuator, thereby causing the controlled electric field to be emitted from the electric field-emitting surface and toward the microbe-containing surface, and wherein the electric field causes irreversible permeabilization of a cell membrane of microbes on the microbe-containing surface.

4. An apparatus for emitting a controlled electric field onto a microbe-containing surface, comprising:
a control circuit, connectable to a power source, and an AC power generating component, wherein the control circuit receives an input electric current from the power source transforms the input electric current into an output electric current having a fundamental frequency; and
an emitter connector component, for receiving current from the control circuit, and delivering it to at least one emitter for emitting an electric field, wherein the control circuit transmits the output electric current from the emitter connector to the emitters at a fundamental frequency in the range from 10 KHz to 200 KHz and subject to over-current control, thereby causing a controlled electric field to be emitted from the emitters with a defined waveform, sufficient to cause irreversible permeabilization of a cell membrane of microbes on a microbe-containing surface proximate to the head component.

5. The apparatus of claim 4, wherein the emitter connector connects to an array of emitters mounted on a flexible substrate.

6. The apparatus of claim 5, wherein the flexible substrate is a surface on a glove.

7. The apparatus of claim 4, wherein the emitter connector connects to a head component comprising a field transport layer that facilitates delivery of the electric field to the microbe-containing surface.

8. The apparatus of claim 7, wherein the field transport layer comprises a material resilient and deformable to follow contours of the microbe-containing surface.

9. The apparatus of claim 7, wherein the field transport layer comprises a wiping cloth removably attached to the head component.

10. The apparatus of claim 7, wherein the field transport layer comprises a material porous and capable of holding a cleaning solution.

11. The apparatus of claim 7, wherein the field transport layer comprises a colloid with a permittivity of 30 or greater.

12. The apparatus of claim 7, wherein the field transport layer comprises a hydrocolloid with a permittivity of 30 or greater.

13. The apparatus of claim 7, wherein the field transport layer comprises a material resilient and deformable to follow contours of the microbe-containing surface with a friction-reducing outer layer.

14. The apparatus of claim 7, wherein the field transport layer comprises a material resilient and deformable to follow contours of the microbe-containing surface with a friction-reducing outer layer that comprises a wiping cloth.

15. The apparatus of claim 5, wherein the field transport layer comprises a material resilient and deformable to follow the contours of the microbe-containing surface.

16. The apparatus of claim 4, wherein the head component comprises stand-off projections to separate the array of emitters from direct contact with the microbe-containing surface, said projections being made of a low friction material.

17. The apparatus of claim 16, wherein the stand-off projections are positioned at the periphery of the head component and the low friction material is a hard, low friction resin.

18. The apparatus of claim 17, wherein the hard, low friction resin is selected from the group consisting of a nylon resin and acetal.

19. The apparatus of claim 4, wherein the emitter connector detachably connects to a component to be treated for microbes, said component being capable of functioning as an emitter so as to deliver the controlled electric field essentially simultaneously to all points on the component.

20. The apparatus of claim 19, wherein the component to be treated for microbes is a working surface.

21. The apparatus of claim 19, wherein the component to be treated for microbes is a cover layer for a working surface.

22. The apparatus of claim 19, wherein the component to be treated for microbes is a curtain.

23. A method for killing microbes comprising:
providing an electrically conductive emitter for emitting an electric field for killing microbes in contact with or in close proximity to the emitter; and
providing a control circuit for electrical connection to the emitter to deliver a current with an AC pulse waveform having a fundamental frequency in the range of 10 KHz to 200 Hz; said control circuit being activated to deliver the current for a defined interval, causing the emitter to emit an electric field sufficient to cause electroporation of microbes in contact with or in close proximity to the emitter, said current being controlled to a level that limits arcing from the emitter to adjacent objects.

24. The method of claim 23 wherein the step of providing the emitter comprises providing an emitter selected to conform to a surface to be treated.

25. The method of claim 23 wherein the step of providing the emitter comprises providing an emitter that is conformable into intimate contact with a portion of a surface to be treated.

26. The method of claim 23 wherein the step of providing the emitter comprises providing an emitter consisting of an array of separate emitters on a substrate conformable into intimate contact with a portion of a surface to be treated.

27. The method of claim 23, wherein the step of providing the emitter comprises providing an emitter comprising a conductive portion that is deformable.

28. The method of claim 23, wherein the step of providing the emitter comprises providing an emitter comprising a conductive portion and a deformable field transport layer with a relatively high permittivity.
